(19) United States
(12) Patent Application Publication
(10) Pub. No.: US 2024/0189565 A1
(43) Pub. Date: Jun. 13, 2024

(54) MICRONEEDLES FOR DELIVERING SELF-ADMINISTRABLE MEDICINE

(71) Applicant: Roxana Belciu Kerns, West Palm Beach, FL (US)

(72) Inventor: Roxana Belciu Kerns, West Palm Beach, FL (US)

(21) Appl. No.: 18/434,721

(22) Filed: Feb. 6, 2024

Related U.S. Application Data
(63) Continuation-in-part of application No. PCT/EP2023/079357, filed on Oct. 20, 2023.
(60) Provisional application No. 63/486,015, filed on Feb. 20, 2023.
(60) Provisional application No. 63/386,640, filed on Dec. 8, 2022.

(51) Int. Cl. A61M 37/00; A61K 9/00

(52) U.S. Cl. CPC A61M 37/0015; A61K 9/0021; A61M 2037/0023; A61M 2037/003; A61M 2037/0046; A61M 2037/0061; A61M 2202/0007; A61M 2202/04; A61M 2202/064; A61M 2202/07; A61M 2205/0238; A61M 2205/3303; A61M 2205/3327; A61M 2205/3561; A61M 2207/10; A61M 2210/04; A61M 2230/201

(57) ABSTRACT

A system for delivering a medication in a liquid suspension, powder or gel through an epidermis of a patient. The system includes: a) a plurality of microneedles which are dissolvable and configured for insertion into a dermis of the patient and delivery into the dermis and/or even further layers of the patient's skin, wherein the plurality of microneedles include: i) one or more neurotoxins and/or one or more dermal fillers; ii) one or more carriers; iii) optionally, one or more local anesthetics; b) one or more substrates configured to be temporarily applied onto the epidermis of the patient, wherein the one or more substrates carry the plurality of microneedles thereon; c) optionally, one or more trays affixed to the one or more substrates, wherein the one or more trays include one or more adhesive patches; and d) optionally, one or more removable covers.

### CROSS-REFERENCE TO RELATED APPLICATION

This application is a continuation-in-part of PCT application no. PCT/EP2023/079357, filed on Oct. 20, 2023, which claims priority to U.S. provisional application Ser. No. 63/486,015, filed on Feb. 20, 2023 and to U.S. provisional application Ser. No. 63/386,640, filed on Dec. 8, 2022, the complete disclosures of which, in their entireties, are herein incorporated by reference.

### TECHNICAL FIELD

The embodiments herein generally relate to a self-administered botulinum toxin purified protein (BTPP) through prefilled frozen microneedle trays. More specifically, the embodiments herein relate to a disposable medical device used in cosmetics, and also pain management, procedures to administer transdermal and/or intramuscular quantitative units of botulinum toxin purified protein within specific dimensions of frozen 0.9% or similar (preservative free) saline microneedle shaped trays.

### BACKGROUND

Within this application there are several patents and publications that are referenced. The disclosures of all these patents and publications, in their entireties, are hereby expressly incorporated by reference into the present application.

The recent Covid-19 pandemic brought new challenges and reinforced current challenges to the cosmetic industry and especially to patients, medical professionals, and pharmaceutical companies involved in the triangle of botulinum toxin purified protein (BTPP) administration or simply botulinum toxin (“BT”) and hyaluronic acid (“HA”) fillers. Due to pandemic business restrictions, patients were unable to receive medical procedures deemed as cosmetic or optional. Thus, what is needed is a means of permitting application of BTPP and injectable HA without the need for a patient to be in the physical presence of a medical professional or medical setting, thus allowing flexibility for the future and providing both patients and professionals with more options.

Currently, botulinum toxin purified protein is typically stored as lyophilized powder in 100 units per glass vial. This powder must be kept at certain temperatures to prevent inactivation. The powder is then diluted prior to application to a patient. Dilution occurs onsite by medical staff and may be prone to human error. What is needed is a means for reducing and even preventing dilution errors such as to deliver an accurate amount of the BTPP.

Today, BTPP and HA are typically delivered to a patient via a hypodermic needle. The hypodermic needles available currently in the industry are essentially metal (e.g., stainless steel) or plastic (e.g., liquid crystal polymer). These hypodermic needles encounter several limitations. They often require (re) sterilization, a process that removes, kills or deactivates all forms of life. The needles may be completely or partially disposable, causing excess waste and requiring specific waste procedures (e.g., medical sharps disposal). The hypodermic needles available need additional, skilled assistance for instrumentation, such as a trained and skilled medical assistant or nurse which can be quite costly and not time efficient. The hypodermic needles require multiple instrumentations into a skin or muscle of a patient to deliver one or more medications, therefore, increasing risk for trauma, bruising, infection increasing handling time, and sometimes requiring the need of analgesic.

Polymeric microneedles (“PMN”) provide a promising solution but are limited. They have been found to have the following challenges: “irritability of the skin, contamination on the micro-level and a lack of mechanical ruggedness in biomaterials, the volume of drug to be loaded on and in the PMN, and the delivery of hydrophilic macromolecules. Consequently, in the absence of accurate mathematical models of the drug release, determining the viability from the assortment of MNs available for targeting numerous diseases seems demanding and exacting” (Gera, A., et al., “The Rise of Polymeric Microneedles: Recent Developments, Advances, Challenges, and Applications with Regard to Transdermal Drug Delivery,” Journal of Functional Biomaterials, Vol. 13, No. 81, Jun. 15, 2022, pp. 1-18).

Polymers are used in the production of dissolving, biodegradable, hydrogel forming, solid, hollow, and coated microneedle (“MN”) arrays. Hollow and porous polymeric microneedles may be beneficial in temporarily securing a microneedle device to a biological barrier (e.g., epidermis) while allowing for drug delivery at better rates than dermal patches and with less pain and less local damage than typical hypodermic needles. For example, see U.S. Pat. No. 8,708,966, incorporated herein by reference in its entirety. But yet, these polymeric microneedles may be prone to breakage and/or bending at the tips. Due to their hollow shaft, these polymeric microneedles may be further weakened. These microneedles may encounter the most resistance at the epidermis, specifically at the stratum corneum. Being inserted into the outermost layer of the epidermis may result in the most damage to the microneedles and inconsistent medication delivery into the epidermis and dermis.

Recently, ice microneedles have come to be studied. “Ice microneedles were used to carry heparin, erythropoietin, as well as biosafe Bacillus subtilis (B. subtilis) for anticoagulation, treating anemia or fungal infection mouse models. All of these results indicated that the ice microneedles could be ideal candidates for practical and universal transdermal delivery.” (Zhang, X., et al., “Versatile Ice Microneedles for Transdermal Delivery of Diverse Actives,” Advanced Science, Vol. 8, Issue 17, Sep. 8, 2021, pp. 1-9).

U.S. Pat. No. 8,420,105 issued to Patricia S. Walker on Apr. 16, 2013 mentions botulinum toxin administered with a carrier transdermal possible ice-crystal. While the conventional solutions may have addressed some of the needs for which they were developed, there remains a need for a new technique for self-administering BTPP.

### SUMMARY

In view of the foregoing, an embodiment herein provide a self-administered prefilled BTPP tray (may be referred to as BTPP tray or tray hereinafter). The BTPP tray may function to solve the challenges of traditional BTPP administration by providing for safe administration under remote (telemedicine) medical guidance of patch like (peel off) frozen 0.9% or similar preservative free saline microneedle trays (1-12 or even more microneedles per tray) filled with quantitative (2-10 or more) units per microneedle.

Other aspects besides correct dilution and ease of use are: saved time spent by medical professionals (no need to mix BTPP powder and reduced costs), saved travel time to a professionals office by a patient, no need for trained medical assistants in the office setting or booking appointments (telemedicine appointments can be directly booked in a doctor's online calendar) therefore better revenue for the medical professional. Less costs for patients and a standardized price for the cosmetic applications of BTPP are also aspects of the techniques provided by the embodiments herein. Patient loyalty is preserved for each medical office as traveling or relocating doesn't necessarily imply a switch in the medical professional. Self-application under controlled remote medical supervision is a breakthrough in BTPP administration that can be extended to other medications. Given the sterile preparation of the product(s) and sterile and safe guided administration, the trauma and infection or bruising associated with BTPP administration while using hypodermic needles is gone. Another aspect of the embodiments herein is the mitigation of pain when compared to that of a traditional hypodermic injection.

The embodiments herein provide for a method of self-administration and prepackaging of BTPP into frozen microneedle trays with adhesive peel off patches. The embodiments herein provide for an improved shape of the frozen medicated composite for deeper skin penetration (e.g., spiral but other shapes can be considered as well). Two to three or even more different sizes and shapes (e.g., pencil point, spiral, quincke, atraucan, sprotte-like, etc.) frozen microneedles, incorporating saline and BTPP can be grouped in a self-adhesive patch to be peeled of and applied to one targeted skin area (e.g. corrugator muscle). This allows different skin penetration and dissolution of the material, avoiding potential side effects (for example, a shorter microneedle targeting the corrugator supercilis muscle while a longer microneedle will deliver BTPP at procerus level or vice-versa).

The embodiments herein provide for a delivery system which is relatively inexpensive to fabricate and exhibits increased utility compared to conventional BTPP administration methods. The embodiments herein provide a unique method of prepackaging of BTPP in self-adhesive trays of frozen 0.9% saline as microneedles of different shapes and sizes. The embodiments herein allow users to self-administer BTPP without trauma or pain, without extraordinary skilled training, without need for re-sterilization, and without requiring a complex operating surgical environment (medical office, hospital, or ambulatory surgical center), in a time efficient and safe way.

The embodiments herein are unique compared to the conventional solutions by providing a) a predetermined dilution of saline and BTPP per microneedle, b) a predetermined depth of penetration, c) most of the microneedle as opposed to just the tip may be dissolved in the target tissue, and d) the ability to provide for individual microneedle insertion or a patch line or even a grouping as prescribed.

The embodiments herein further provide a system for delivering one or more medications through or into an epidermis of a patient, wherein the system includes: a) a plurality of microneedles which are dissolvable and configured for insertion into a dermis (i.e., via insertion through an epidermis) of the skin and delivery into the dermis and/or even further layers of the patient's skin, wherein the plurality of microneedles include: i) one or more neurotoxins, recombinant neurotoxin and/or one or more dermal fillers; ii) one or more reconstitution sterile fluid and or stabilizing agents for the neurotoxin (carriers); iii) optionally, one or more local anesthetics; b) one or more substrates configured to be temporarily applied onto the epidermis of the patient, wherein the one or more substrates carry the plurality of microneedles thereon.

The embodiments herein further provide a system for delivering one or more medications through or into an epidermis of a patient, wherein the system includes: a) a plurality of microneedles which are dissolvable and configured for insertion into a dermis (i.e., via insertion through an epidermis) of the patient's skin and delivery into the dermis and/or even further layers of the skin, wherein the plurality of microneedles include: i) one or more neurotoxins, recombinant neurotoxin and/or one or more dermal fillers; ii) one or more reconstitution sterile fluid; iii) optionally, one or more local anesthetics; b) one or more substrates configured to be temporarily applied onto the epidermis of the patient, wherein the one or more substrates carry the plurality of microneedles thereon; c) optionally, one or more trays affixed to the one or more substrates on an opposite side as the plurality of microneedles, wherein the one or more trays include one or more adhesive patches; and d) optionally, one or more removable covers which are removably adhered to the one or more substrates and/or one or more trays.

Another embodiment provides a system for delivering one or more medications through an epidermis of a subject intradermal or subdermal, wherein the system comprises: a tray, and one or more substrates arranged on the tray, the substrates carrying one or more microneedle assemblies, wherein the one or more microneedle assemblies comprise one or more microneedles configured as a plurality of overlapping 1-6 layers of a combination of non-nested and nested microneedles, wherein a nested microneedle comprises one microneedle resting partially within another microneedle, wherein the one or more microneedle assemblies are dissolvable frozen and/or polymeric and are to be inserted into a dermis or muscle of the subject via insertion through an epidermis of the skin of the subject, wherein the one or more microneedle assemblies are prepackaged with neurotoxin quantified units with a predetermined reconstitution amount of saline per microneedle to yield a set dilution, and wherein the one or more of the microneedle assemblies carry the one or more medications for delivery of into the dermis and/or further layers of the subject.

The one or more medications may be included in the one or more microneedles as coated tips, lyophilized powder laser engraved in a base, tip, or shaft of the one or more microneedles, or embedded in micro spheres of poly-(L-amide) or cross-linked hyaluronic acid within a hollow portion of a body of the one or more microneedles. The nested microneedle may comprise a nesting cavity that at least partially receives and/or retains a subsequent microneedle inserted therein. The one or more microneedles may comprise: one or more neurotoxins or recombinant neurotoxin; and preservative free saline. Each of the one or more microneedles may comprise a lyophilized powder or quantified Botulinum toxin purified protein type A (BTTP) and a preservative free, frozen saline. The one or more microneedles may be free of any additional carrier separate from a frozen saline. The one or more microneedles may comprise one or more local anesthetics selected from the group consisting of lidocaine, prilocaine, mepivacaine. The one or more microneedles may comprise a base comprising one or more adhesive peel off patches. The one or more microneedles may comprise a hollow interior containing one or more of the medications.

The one or more microneedles may comprise a reservoir containing one or more of the medications, wherein the reservoir is in communication with a hollow shaft of a corresponding microneedle and/or with the one or more substrates, and wherein the reservoir is to propel BTTP embedded micro-spheres through fluidic force and/or a pressure leading microneedle through a stratum corneum and intermediate or trailing microneedles through created micropores.

The one or more microneedle assemblies may comprise a leading microneedle, one or more intermediate microneedles, and one or more trailing microneedles, or a combination thereof, wherein the one or more intermediate microneedles is nested in the leading microneedle, and wherein the one or more trailing microneedles is nested in the one or more intermediate microneedles. One aspect of using nested microneedles over non-nested microneedles relates to the possibility of having dissolvable microneedles nesting with hollow microneedles or hydrogels like microneedles. In this manner the leading microneedle can be dissolvable, the intermediate microneedle can be hydrogel forming, and the trailing microneedle hollow connected to the reservoir.

The leading microneedle may be triangular shape, the one or more intermediate microneedles may be pyramidal shape, and the one or more trailing microneedles may be conical shape. The tip of the one or more intermediate microneedles and/or the one or more trailing microneedles may be embedded in a base of the leading microneedle. The one or more medications in frozen microneedles may form a needle structure without any additional carriers. The mechanical resistance of the one or more intermediate microneedles and the one or more trailing microneedles may be less than the mechanical resistance of the leading microneedle. The microneedles of the one or more microneedle assemblies may progressively increase in width and/or length from the leading microneedle to the one or more trailing microneedles, as a whole along an entire height of a connected assembly, and wherein each of the nested microneedles is shorter than an overall combination of overlapping microneedles. The one or more microneedles may be provided as a plurality of microneedles and arranged in one or more arrays, groups, random patterns, or a combination thereof on the tray, the one or more substrates, or a combination thereof.

Another embodiment provides a method of delivering a medicament in liquid suspension, wherein the method comprises: removing one or more adhesive patches from a tray, wherein the one or more adhesive patches carry one or more microneedles; applying the one or more adhesive patches containing the one or more microneedles to an epidermis of a subject, wherein the one or more microneedles comprise a plurality of overlapping 1-6 layers of a combination of non-nested and nested microneedles, and wherein a nested microneedle comprises one microneedle resting partially within another microneedle; inserting, into a dermis of the subject, the one or more microneedles formed of the liquid suspension containing the medicament, wherein the one or more microneedles are in a frozen form and/or polymeric upon insertion; and the one or more microneedles melting after insertion into the dermis.

The method may comprise applying the one or more patches in an overlapping pattern on the epidermis of the subject. The method may comprise applying pressure to the one or more microneedles upon insertion in the dermis of the subject, wherein a first layer of a leading microneedle dissolves after insertion and forms a plurality of microchannels in the dermis. The method may comprise continuing to apply pressure to the one or more microneedles, wherein subsequent layers of intermediate microneedles or trailing microneedles advance through the microchannels. The method may comprise reapplying additional microneedles in the dermis after dissolution of the one or more microneedles. The non-nested microneedles may be configured to (i) determine glucose or viral RNA levels at an intercellular level, through a continuous sensor and transmitter incorporated within a substrate of a non-nested microneedle; (ii) transmit information about the glucose or viral RNA levels to a receiver incorporated in another substrate of another non-nested microneedle, on the tray that supports nested microneedles with targeted medications comprising short-acting insulin, long-acting insulin, or Covid-19 antibodies.

Another embodiment provides a method of manufacturing one or more dissolvable polymeric and/or frozen microneedles, the method comprising 3D printing one or more dissolvable polymeric and/or frozen microneedles made of hyaluronic acid (HA) cross-linked with medium and low molecular weight as methacrylic anhydride cross linked hyaluronic acid (MeHA); filling the one or more dissolvable polymeric and/or frozen microneedles with one or more medications; and performing: laser edging to engrave one or more neurotoxins that are either in a powder or liquid form into a base, tip, or shaft of the one or more dissolvable polymeric and/or frozen microneedles; covering bases of the one or more dissolvable polymeric and/or frozen microneedles with one or more substrate; or filling frozen saline pre-shaped microneedles with neurotoxin in a matrix, as embedded microspheres or coated tips.

The embodiments herein may be useful with delivery of botulinum toxin purified protein (BTPP), hyaluronic acid (HA) various cross-linking, saline, lidocaine, insulin, and/or anti-RNA antibodies, or the like. The rate of drug release and mechanical strength can be controlled by adjusting the cross-linking density of HA. HA as a polymer for MN fabrication is classified into three types with the sustained drug releasing HA having the strongest mechanical strength and loading drug capacity. Cross-linking with MeHA(methacrylic anhydride) can stabilize peptides.

The embodiments herein provide microneedles which may be dissolvable. Dissolvable microneedles may be frozen microneedles, polymeric microneedles, or both. The embodiments herein further provide a method of applying and delivering the one or more medications of the system described. The system may provide a more effective method for self-administering botulinum toxin at home by providing a more mechanically rugged microneedle, capable of penetrating the epidermis without causing irritation of the skin and reducing micro-level contamination upon penetration. The system may provide a system which is at least partially dissolvable or entirely dissolvable, thus eliminating the disposal procedures associated with medical “sharps”. The system can be applied by a patient without the need for medical staff presence.

These and other aspects of the embodiments herein will be better appreciated and understood when considered in conjunction with the following description and the accompanying drawings. It should be understood, however, that the following descriptions, while indicating exemplary embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a schematic diagram illustrating a system according to an embodiment herein;

FIG. 2 is a schematic diagram illustrating another system according to an embodiment herein;

FIG. 3 is a schematic diagram illustrating another system according to an embodiment herein;

FIG. 4 is a flowchart of a method of use of the microneedle tray of any of FIGS. 1 to 3, according to an embodiment herein;

FIG. 5 is a schematic diagram illustrating another system according to an embodiment herein;

FIG. 6A is a schematic diagram illustrating a configuration for a nested microneedle according to an embodiment herein;

FIG. 6B is a schematic diagram illustrating another configuration for a nested microneedle according to an embodiment herein;

FIG. 6C is a schematic diagram illustrating another configuration for a nested microneedle according to an embodiment herein;

FIG. 6D is a schematic diagram illustrating another configuration for a nested microneedle according to an embodiment herein;

FIG. 6E is a schematic diagram illustrating another configuration for a nested microneedle according to an embodiment herein;

FIG. 7 is a schematic diagram illustrating another system according to an embodiment herein;

FIG. 8 is a schematic diagram illustrating another system according to an embodiment herein;

FIG. 9 is a schematic diagram illustrating another system according to an embodiment herein;

FIG. 10 is a schematic diagram illustrating another system according to an embodiment herein;

FIG. 11 is a schematic diagram illustrating another system according to an embodiment herein;

FIG. 12 is a schematic diagram illustrating another system according to an embodiment herein;

FIG. 13A is a flow diagram illustrating a method of applying one or more medications according to an embodiment herein;

FIG. 13B is a flow diagram illustrating a method of preparing a patient's skin for applying one or more medications according to an embodiment herein;

FIG. 13C is a flow diagram illustrating a method of preparing a system for applying one or more medications according to an embodiment herein;

FIG. 14A is a flow diagram illustrating a method of manufacturing one or more dissolvable polymeric microneedles according to an embodiment herein;

FIG. 14B is a flow diagram illustrating a further method of manufacturing one or more dissolvable polymeric microneedles according to an embodiment herein;

FIG. 15A is a flow diagram illustrating a method of manufacturing one or more frozen microneedles according to an embodiment herein;

FIG. 15B is a flow diagram illustrating a further method of manufacturing one or more frozen microneedles according to an embodiment herein;

FIG. 16A is block diagrams illustrating a system for delivering one or more medications through an epidermis of a subject intradermal or subdermal according to an embodiment herein;

FIG. 16B is a block diagram illustrating aspects of the microneedles of the system of FIG. 16A according to an embodiment herein;

FIG. 16C is a block diagram illustrating further aspects of the microneedles of the system of FIG. 16A according to an embodiment herein;

FIG. 16D is a block diagram illustrating still further aspects of the microneedles of the system of FIG. 16A according to an embodiment herein;

FIG. 16E is a block diagram illustrating aspects of the nested and non-nested microneedles of the system of FIG. 16A according to an embodiment herein; and

FIG. 17 is a flow diagram illustrating a method of manufacturing one or more dissolvable polymeric and/or frozen microneedles according to an embodiment herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

The explanations and illustrations presented herein are intended to acquaint others skilled in the art with the embodiments herein, its principles, and its practical application. The specific embodiments of the embodiments herein as set forth are not intended as being exhaustive or limiting of the embodiments herein. The scope of the embodiments herein should be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein. The following description of particular embodiment(s) is merely exemplary in nature and is in no way intended to limit the scope of the invention, its application, or uses, which can, of course, vary.

It will be understood that when an element or layer is referred to as being “on”, “connected to”, or “coupled to” another element or layer, it may be directly on, directly connected to, or directly coupled to the other element or layer, or intervening elements or layers may be present. In contrast, when an element or layer is referred to as being “directly on”, “directly connected to”, or “directly coupled to” another element or layer, there are no intervening elements or layers present. It will be understood that for the purposes of this disclosure, “at least one of X, Y, and Z” or “any of X, Y, and Z” may be construed as X only, Y only, Z only, or any combination of two or more items X, Y, and Z (e.g., XYZ, XY, XZ, YZ).

The description herein describes inventive examples to enable those skilled in the art to practice the embodiments herein and illustrates the best mode of practicing the embodiments herein. Upon reading the following description in light of the accompanying drawing figures, those skilled in the art will understand the concepts of the disclosure and will recognize applications of these concepts not particularly addressed herein.

The terms first, second, etc. may be used herein to describe various elements, but these elements should not be limited by these terms as such terms are only used to distinguish one element from another. For example, a first element could be termed a second element, and, similarly, a second element could be termed a first element, etc. without departing from the scope of the present disclosure. As used herein, the term “and/or” includes any and all combinations of one or more of the associated listed items.

Furthermore, although the terms “final”, “first”, “second”, “upper”, “lower”, “bottom”, “side”, “intermediate”, “middle”, and “top”, etc. may be used herein to describe various elements, but these elements should not be limited by these terms. These terms are only used to distinguish one element from another. For example, a first element could be termed an “top” element and, similarly, a second element could be termed a “top” element depending on the relative orientations of these elements.

The terminology used herein is for the purpose of describing particular embodiments only and is not intended to be limiting of the disclosure. As used herein, the singular forms “a”, “an”, and “the” are intended to include the plural forms as well, unless the context clearly indicates otherwise. Accordingly, the use of “a” or “one” to describe an element, ingredient, component, or step is not intended to foreclose additional elements, ingredients, components, or steps. “Or” means “and/or.” As used herein, the term “and/or” includes any and all combinations of one or more of the associated listed items. Plural elements, ingredients, components, or steps can be provided by a single integrated element, ingredient, component, or step. Alternatively, a single integrated element, ingredient, component, or step might be divided into separate plural elements, ingredients, components, or steps.

It will be further understood that the terms “comprises” and/or “comprising,” or “includes” and/or “including” when used herein, specify the presence of stated features, regions, integers, steps, operations, elements, and/or components, but do not preclude the presence or addition of one or more other features, regions, integers, steps, operations, elements, components, and/or groups thereof. Accordingly, the use of the terms “comprising” or “including” to describe combinations of elements, ingredients, components, or steps herein also contemplates embodiments that consist essentially of the elements, ingredients, components, or steps. The term “or a combination thereof” means a combination including at least one of the foregoing elements. The term “consisting essentially of” to describe a combination shall include the elements, ingredients, components, or steps identified, and such other elements ingredients, components or steps that do not materially affect the basic and novel characteristics of the combination.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Any numerical values recited herein include all values from the lower value to the upper value in increments of one unit provided that there is a separation of at least two units between any lower value and any higher value. These are only examples of what is specifically intended and all possible combinations of numerical values between the lowest value, and the highest value enumerated are to be considered to be expressly stated in this application in a similar manner. Unless otherwise stated, all ranges include both endpoints and all numbers between the endpoints.

The terms “generally” or “substantially” to describe angular measurements may mean about +/−10° or less, about +/−5° or less, or even about +/−1° or less. The terms “generally” or “substantially” to describe angular measurements may mean about +/−0.01° or greater, about +/−0.1° or greater, or even about +/−0.5° or greater. The terms “generally” or “substantially” to describe linear measurements, percentages, or ratios may mean about +/−10% or less, about +/−5% or less, or even about +/−1% or less. The terms “generally” or “substantially” to describe linear measurements, percentages, or ratios may mean about +/−0.01% or greater, about +/−0.1% or greater, or even about +/−0.5% or greater.

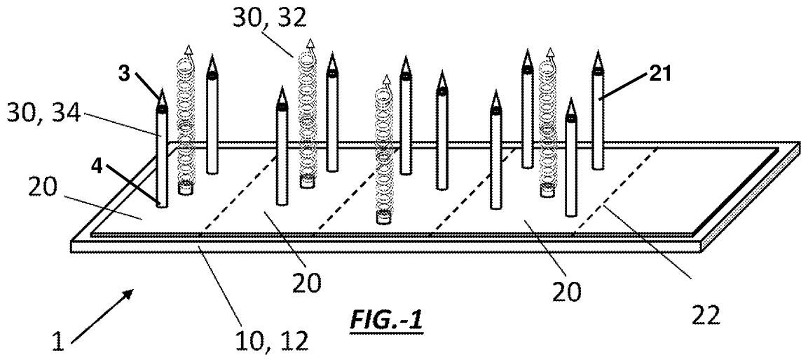

Referring now to the drawings, and more particularly to FIGS. 1 through 17, where similar reference characters denote corresponding features consistently throughout the figures, there are shown preferred example embodiments. In the drawings, the size and relative sizes of components, layers, and regions, etc. may be exaggerated for clarity. The figures are provided examples of the embodiments herein but are not intended to limit the scope thereof.

FIGS. 1 through 12 illustrate a system 1 and method 200 for delivering one or more medications (e.g., a medicament 21) in liquid suspension (powder, viscous) through or into an epidermis of a patient, wherein the system 1 includes (a) one or more frozen microneedles 30 formed of the liquid suspension in frozen form and which are dissolvable and configured for insertion into a dermis (i.e., via insertion through an epidermis) of the patient's skin and then subsequent melting and delivery into the dermis or even further layers of the patient's skin; (b) one or more adhesive patches 20 configured to be temporarily applied onto the epidermis of the patient, wherein the adhesive patch 20 carries the one or more (layers of) frozen microneedles 30 thereon; and (c) a tray 10 which is removably adhered to the adhesive patch 20 on an opposite side as the one or more frozen microneedles 30. The medicament 21 may be botulinum toxin purified protein. The medicament 21 may be used for cosmetic, pain management (e.g., trigger point injection), and/or other therapeutic purposes (e.g., diabetic treatment, vaccines, etc.). The tray 10 may carry a single or a plurality of the adhesive patches 20 thereon. The one or more adhesive patches 20 may each carry a single or a plurality of the frozen microneedles 30. The one or more frozen microneedles 30 may be configured with a shape which is substantially a pencil point, spiral, quincke, atraucan, sprotte, triangular, conical, pyramidal the like, or different, or any combination thereof.

The one or more microneedles 30 include: i) one or more neurotoxins, recombinant neurotoxin, and/or one or more dermal fillers; ii) one or more reconstitution sterile fluid and/or stabilizing agents for the neurotoxin; iii) optionally, one or more local anesthetics; b) one or more substrates configured to be temporarily applied onto the epidermis of the patient, wherein the one or more substrates carry the one or more microneedles thereon; c) optionally, one or more trays 10 affixed to the one or more substrates on an opposite side as the one or more microneedles 30, wherein the one or more trays 10 include one or more adhesive; and d) optionally, one or more removable covers which are removably adhered to the one or more substrates and/or one or more trays 10. Herein, “substrate” and “patch” or “patches” may be used interchangeably.

Each of the one or more frozen microneedles 30 may be prepackaged with a lyophilized powder of quantified Botulinum toxin purified protein (x) units and a corresponding amount (to yield a set dilution) of preservative free 0.9% or similar saline frozen (at predetermined temperatures which preserve the integrity and function of the Botulinum toxin purified protein). The one or more frozen microneedles 30 may be free of any additional carrier separate from a frozen saline (i.e., to maintain an accurate dilution). The shape, depth, and/or size of the one or more microneedles 30 may be selected based on a target intradermal and/or subdermal location of a patient. The tray 10 and/or each individual adhesive patch 20 may have one or more frozen microneedles 30 provided as a plurality of frozen microneedles 30 in a plurality of differing shapes.

The differing shapes may include two to three or even more shapes and/or sizes. The one or more frozen microneedles 30 may be provided as a plurality of frozen microneedles 30 which are configured in one or more arrays, groups, random patterns, the like, or any combination thereof on the tray 10, the one or more adhesive patches 20, or both. The one or more frozen microneedles 30 may be configured such as to entirely, or mostly, penetrate through an epidermis and allow mixing of composite at target level as frozen saline becomes fluid at body temperature. The system 1 may be configured for safe patient self-administration of the tray 10 of one more microneedles 30 under virtual and/or remote medical (tele)guidance. The one or more trays 10 include one or more adhesive patches 20.

The one or more frozen or polymeric microneedles 30, 34 may include a plurality of nesting (overlapping) microneedles with increasing width and length from the skin (top) layer on. The plurality of nesting microneedles may include a leading microneedle 42, one or more intermediate microneedles 44, one or more trailing microneedles 46, or a combination thereof. The nesting (leading) microneedles 42 may each have a height of about 50 microns to about 1,700 microns. The nesting (e.g., intermediate, trailing) microneedles 44, 46 may each have a height of about 100 microns to about 2000 microns. At least one of the nesting microneedles may be free of medicament 21.

At least one of the nesting microneedles may include a medicament 21 which includes anesthetic, botulinum toxin, hyaluronic acid dermal filler, insulin, and/or anti-RNA antibodies, or the like, or any combination thereof. The microneedles 30 may be frozen saline, (hyaluronic acid (HA) fillers) polymeric, or a combination thereof. The HA filler may be of different viscosities and cross linking (e.g., Juvederm Ultra® or Juvederm Voluma®). The nesting microneedles may be formed such that a tip 3 of an intermediate microneedle 44 and/or trailing microneedle 46 (from a subsequent layer) is embedded in a base 4 of a leading microneedle 42 (top layer). The shape, thickness, length, and/or a mechanical resistance of a leading microneedle 42 may follow Young's Modulus to ensure penetration through the stratum corneum. The intermediate microneedle 44, trailing microneedle 46, or both can be weaker (less mechanical resistance) than the leading microneedle 42.

Again, the system 1 may include one or more microneedles 30, which may function to pierce through the epidermis, dermis, and/or even further into muscle of the patient. One or more microneedles 30 may form one or more micro channels and/or micro pores into the epidermis of the skin. One or more microneedles 30 may deliver or be free of delivering one or more medications (medicament 21) into the epidermis, dermis, and/or muscle of the patient. The plurality of microneedles 30 may cooperate together to pierce deeper into a patient as compared to the capability of a single microneedle or a single layer of one or more microneedles, delivering medication simultaneously over a wider area of a patient, or both. One or more microneedles 30 may be affixed to, integral with, or both a substrate, a tray, or both.

One or more microneedles 30 may have one or more shapes. The one or more shapes may be a spiral, triangular, pyramidal, conical, pencil point, quincke, atraucan, sprotte-like, and/or another shape. The one or more shapes may include a shape, a depth, a size, a length, a width, a circumference, a slope angle, a base dimension, a tip radius or any combination thereof.

The one or more shapes may include only one shape for each of the one or more microneedles 30, different shapes for each of the one or more microneedles 30, or any mix of the one or more microneedles 30 having similar shapes or dissimilar shapes. The shape, depth, and/or size of the one or more microneedles 30 may be elected based on a target intradermal and/or subdermal location of a patient. One or more shapes may include multiple shapes to target multiple intradermal and/or subdermal locations of a patient. The one or more shapes may allow complete or almost complete skin penetration. The one or more shapes may allow for minimal force to be applied to achieve skin penetration. For example, a microneedle 30 can be thinner at the tip 3 and wider/thicker at the base, have a small tip radius, or both. The base 4 may be adjacent, closest to, affixed to, or integral with a tray 10 and/or substrate (e.g., patch 20). The tip 3 may be opposite the base 4, opposite a tray 10 and/or substrate (e.g., patch 20), the leading point of a microneedle 30, or any combination thereof. Such shape may allow entire dissolution of material at the target intradermal and/or subdermal location of a patient by hand application. The shape of the one or more microneedles 30 may be an external shape where the one or more microneedles 30 have a hollow interior. One or more microneedles 30 may be formed as dissolvable. Dissolvable means that the one or more microneedles 30 may be formed of one or more materials which are highly water soluble.

Dissolvable may mean that the temperature of the receiving skin may melt or otherwise dissolve the one or more microneedles 30, resulting in delivering their medications (e.g., medicament 21) into the patient's epidermis, dermis, muscles, or the like. Dissolvable may mean that moisture contained in skin is absorbed by the one or more microneedles 30 until the one or more microneedles 30 dissolve due to the moisture. Dissolvability may be achieved via frozen microneedles 30, dissolvable polymeric microneedles 34, or both. Dissolvability may be beneficial in eliminating biohazardous sharps and their associated waste protocols.

One or more microneedles 30 may be formed as one or more dissolvable polymeric microneedles 34. A dissolvable polymeric microneedle 34 may be formed using one or more dissolvable polymers. One or more dissolvable polymers may include any polymer suitable for dissolution into a patient. One or more dissolvable polymers may include natural polymers and/or synthetic polymers. One or more dissolvable polymers may include hyaluronic acid, carboxymethyl cellulose (CMC), hydroxypropyl methyl cellulose (HPMC), sodium alginate, silk and chitosan, polycaprolactone (PCL), polylactic acid (PLA), polyvinyl alcohol (PVA), polyvinyl pyrrolidone (PVP), polylactic coglycolic acid (PLGA), the like, or any combination thereof. One or more dissolvable polymers may form one or more microspheres. One or more dissolvable polymers formed as one or more microspheres may contain one or more medications (e.g., medicament 21). One or more dissolvable polymers formed as one or more microspheres may transfer one or more medications (e.g., medicament 21) through or into an epidermis, dermis, muscle, or other body layer before releasing the one or more medications (e.g., medicament 21). Suitable dissolvable polymeric materials and their formation may be as taught in Sullivan, S., et al., “Dissolving Polymer Microneedle Patches for Influenza Vaccination,” Nat. Med. 2010 August; 16(8), pp. 915-920 and Ali, M., et al., “Dissolvable polymer microneedles for drug delivery and diagnostics,” Journal of Controlled Release, Vol. 347, July 2022, pp. 561-589., which are incorporated herein by reference in their entirety. One or more dissolvable polymeric microneedles 34 may have medication within, on the surface of, or both each dissolvable polymeric microneedle 30.

The one or more microneedles 30 or one or more dissolvable polymeric microneedles 34 may be manufactured using a polymeric microneedle manufacturing method (PMMM). The PMMM may be used to create one or more microneedles 34. The one or more microneedles 34 created in the PMMM may be dissolvable polymeric microneedles 34 or a combination of dissolvable polymeric microneedles 34 and frozen microneedles 30. The PMMM may be used to create one or more substrates (e.g., patches 20). The one or more substrates (e.g., patches 20) created by the PMMM may be formed of a dissolvable polymer. The PMMM may utilize 3D printing, molding, micromolding, or other manufacturing processes. The 3D printing process used by the PMMM may be a two-photon polymerization (TPP) 3D printing process. The TPP 3D printing process may be that disclosed in Islam, H., et al., “3D Printed Hollow Microneedles for Treating Skin Wrinkles Using Different Anti-Wrinkle Agents: A Possible Futuristic Approach,” Cosmetics 2023, Vol. 10(2), No. 41, Feb. 27, 2023, pp. 1-23, and incorporated by reference herein in its entirety. The PMMM may be implemented independent of or in combination with a frozen microneedle manufacturing method (FMMM).

The PMMM may involve multiple steps to create one or more microneedles 30, 34. The first step of the PMMM may be creating a CAD model of the one or more microneedles 30, 34 the one or more substrates (e.g., patches 20), or both. The second step of the PMMM may be 3D printing the one or more microneedles 30, 34. The second step of the PMMM may include 3D printing one or more microneedles 30, 34 which are hollow. The second step of the PMMM may be 3D printing arrays of 10 to 50 of the one or more microneedles 30, 34. The second step of the PMMM may be 3D printing the one or more microneedles 30, 34 with the same shape, different shapes, the same heights, different heights, or any combination thereof. The second step of the PMMM may be 3D printing one or more layers of one or more microneedles 30, 34 including one or more leading microneedles 42, intermediate microneedles 44, trailing microneedles 46, or any combination thereof which may be nesting. The third step of the PMMM may be adding one or more reconstitution sterile fluid and or stabilizing agents for the neurotoxin into the one or more microneedles 30, 34. The third step of the PMMM may be adding one or more reconstitution sterile fluid which may include saline, preservative free 0.9% saline, or one or more other stabilizing agents. The third step of the PMMM may be adding 2.5 ml of one or more reconstitution sterile fluids into the one or more microneedles 30, 34. The third step of the PMMM may include adding one or more medications (e.g., medicament 21) into or onto the one or more microneedles 30, 34. The fourth step of the PMMM may be freezing the one or more microneedles 30. The fourth or fifth step of the PMMM may be freezing the one or more microneedles 30 including one or more reconstitution sterile fluids. The fourth step of the PMMM may be freezing the one or more microneedles 30 to −20° C., a temperature that stabilizes BTPP, a temperature that enhances the one or more microneedles' 30 ability to piece skin, or any combination thereof. The fifth/sixth step of the PMMM may be laser edging (bases 4, along the shaft 48 or tip 3) of the one or more microneedles 30, 34. The laser edging may bind BTPP in powder (lyophilized) or liquid form to the one or more microneedles 30, 34 (powder-laden HA MNP). The following step of the PMMM may be covering bases 4 (where lyophilized botulinum toxin is inserted) of the one or more microneedles 30, 34 with one or more substrates (e.g., patches 20). The seventh step of the PMMM may include affixing a tray 10, an adhesive patch 20 or both to the one or more substrates (e.g., patches 20). The eighth step of the PMMM may include storing the one or more microneedles 30, 34 until use on a patient. The eighth step of the PMMM may include freezing the one or more microneedles 30 below 0° C. to maintain the integrity of the one or more microneedles 30, preserve BTPP, or both. The PMMM may include any combination of steps in any order or include additional steps.

One or more microneedles 30 may be formed as one or more frozen microneedles 30. One or more frozen microneedles 30 may be configured such as to entirely, or partially, penetrate through an epidermis and allowing mixing of one or more medications (e.g., medicament 21) with one or more reconstitution sterile fluids (e.g., saline) to form a composite, and/or release of one or more medications (e.g., medicament 21) at the target level as one or more reconstitution sterile fluids (e.g., saline) and or stabilizing agents which are frozen become fluid at body temperature. The one or more frozen microneedles 30 may be formed as a needle structure. A needle structure may be the one or more materials which form the general shape when frozen (e.g., molded and frozen) of the one or more frozen microneedles 30 and which are dissolvable. The needle structure may be made from saline, botulinum toxin purified protein (“BTPP”), hyaluronic acid (“HA”), other medications (e.g., medicament 21), and/or the like. One such suitable composition is disclosed in Zhang, X., et al., “Versatile Ice Microneedles for Transdermal Delivery of Diverse Actives,” Advanced Science, Vol. 8, Issue 17, Sep. 8, 2021, pp. 1-9, and incorporated by reference herein in its entirety. The embodiments herein may be useful in providing a frozen microneedle 30 which is free of additional carriers, such as water, GelMA, or Alg.

The one or more frozen microneedles 30 may be manufactured using a frozen microneedle manufacturing method (FMMM). The FMMM may be used to create one or more microneedles 30. The one or more microneedles 30 created in the FMMM may be frozen microneedles 30 or a combination of dissolvable polymeric microneedles 34 and frozen microneedles 30.

The FMMM may be used to create one or more substrates (e.g., patches 20). The one or more substrates (e.g., patches 20) created by the FMMM may be formed of one or more reconstitution sterile fluids, one or more medications (e.g., medicament 21), or both. The FMMM may utilize 3D printing, molding, micromolding, or other manufacturing processes. The 3D printing process used by the FMMM may be an ice 3D printing process. The ice 3D printing process may be as disclosed in Garg, A., et al., “Freeform 3D Ice Printing (3D-ICE) at the Micro Scale,” Adv. Sci., Vol. 9, Issue 27, Jul. 6, 2022, pp. 1-11, and incorporated by reference herein in its entirety. The FMMM may be implemented independent of or in combination with a polymeric microneedle manufacturing method (PMMM).

The FMMM may involve multiple steps to create one or more microneedles 30. The first step of the FMMM may be creating a CAD model of the one or more microneedles 30, the one or more substrates (e.g., patches 20), or both. The second step of the FMMM may be 3D printing the one or more microneedles 30. The second step of the FMMM may include 3D printing one or more microneedles 30 which may be hollow. The second step of the FMMM may be 3D printing arrays of 10 to 50 of the one or more microneedles 30. The second step of the FMMM may be 3D printing the one or more microneedles 30 with the same shape, different shapes, the same heights, different heights, or any combination thereof. The second step may include creating one or more microneedles 30 by a molding process. The molding process may involve filling a microneedle mold with one or more reconstitution sterile fluids, one or more medications (e.g., medicament 21), or both and freezing the mold. The third step of the FMMM may be adding one or more reconstitution sterile fluids and or stabilizing agents for the neurotoxin into the one or more microneedles 30. The third step of the FMMM may be adding saline, preservative free 0.9% saline. The third step of the FMMM may be adding 2.5 ml of one or more stabilizing agents or reconstitution sterile fluids into the one or more microneedles 30. The fourth step of the FMMM may be freezing the one or more microneedles 30. The third step of the FMMM may include adding one or more medications (e.g., medicament 21) into or onto the one or more microneedles 30. The fourth step of the FMMM may be freezing the one or more microneedles 30 including one or more reconstitution sterile fluids. The fourth step of the FMMM may be freezing the one or more microneedles 30 to −20° C., a temperature that stabilizes BTPP, a temperature that enhances the one or more microneedles' 30 ability to piece skin, or any combination thereof. The fifth step of the FMMM may be laser edging bases 4, longitudinal shaft 48, or tip 3 of the one or more microneedles 30. The laser edging may bind BTPP in powder (lyophilized) or liquid form to the one or more microneedles 30. The sixth step of the FMMM may be covering bases 4 of the one or more microneedles 30 with one or more substrates (e.g., patches 20). The seventh step of the FMMM may include affixing a tray 10, an adhesive patch 20 or both to the one or more substrates (e.g., patches 20). The eighth step of the FMMM may include storing the one or more microneedles 30 until use on a patient. The eighth step of the FMMM may include freezing the one or more microneedles 30 below 0° ° C. to maintain the integrity of the one or more microneedles 30, preserve FMMM, or both. The FMMM may include any combination of steps in any order or include additional steps.

The one or more frozen microneedles 30 may include as part of its needle structure and/or within a hollow interior: saline, BTPP type A, recombinant neurotoxin, and/or other medications (e.g., insulin, anti-RNA antibodies, medicament 21). It is contemplated that an individual frozen microneedle 30 may contain frozen (−20° C. or other), preservative free 0.9% saline at a set volume (for example, from 0.05 ml), and lyophilized purified protein of botulinum toxin set units (for example, from 2 set units or more). One or more medications (e.g., medicament 21) may include one or more neurotoxins, one or more reconstitution sterile fluids, one or more dermal fillers, or the like. One or more medications (e.g., medicament 21) may be applied to an external surface of, encapsulated within the matrix of, or both the one or more microneedles 30. One or more medications (e.g., medicament 21) may be encapsulated into microspheres of poly-L-lactide or one or more other dissolvable polymers which are embedded into the matrix of one or more microneedles 30, fill a hollow interior of one or more microneedles 30, or both. The skin where one or more frozen microneedles 30 will be applied may be warmed prior to application to facilitate more rapid melting of the one or more frozen microneedles 30. As an alternative, the skin where one or more frozen microneedles 30 may be applied may be cooled to facilitate slower melting of the one or more frozen microneedles 30, thus being more conducive to maintaining the piercing structure at the tip 3 of the one or more microneedles 30.

For the one or more frozen microneedles 30, instead of using a carrier as described in U.S. Pat. No. 8,420,105 or in Zhang, X., et al., “Versatile Ice Microneedles for Transdermal Delivery of Diverse Actives,” Advanced Science, Vol. 8, Issue 17, Sep. 8, 2021, pp. 1-9, embodiments herein may provide a composite of saline, BTPP, and/or HA without the need for additional water or other soluble materials to form the needle structure. In other words, the same medications (e.g., medicament 21) which are intentionally being delivered to the patient may form the dissolvable carrier (e.g., needle structure). The embodiments herein may provide a dissolvable carrier made of a composite of preservative free 0.9% saline (frozen), lyophilized BTPP powder (to be released and mixed up at target level), hyaluronic acid, or any combination thereof in any ratio. No additional water and/or soluble materials may be used as carriers. This may avoid further diluting the concentration of the composite. By avoiding further dilution upon dissolution, this may aid in preventing a change of absorption at the target level. The BTPP may also be embedded into a shaft of one or more dissolvable polymeric microneedles 34 which are hollow, one or more frozen microneedles 30 which are hollow, and/or possibly as coated microneedle tips 3 or other variations.

The one or more microneedles 30, 34 may include one or more medications (e.g., medicament 21). The one or more medications (e.g., medicament 21) may be for cosmetic, pain management (e.g., trigger point injection), and/or other therapeutic purposes. One or more medications (e.g., medicament 21) may include one or more neurotoxins, recombinant neurotoxin, one or more dermal fillers, one or more reconstitution sterile fluids and or stabilizing agents for the neurotoxin, one or more local anesthetics, the like, or any combination thereof.

The one or more microneedles 30, 34 may include one or more neurotoxins. One or more neurotoxins may function to relax muscles; temporarily reduce and/or eliminate facial fine lines, wrinkles, or both; reduce muscle spasms; reduce sweating; reduce or improve migraines; the like; or any combination thereof. The one or more neurotoxins may include any neurotoxins suitable for delivery via the epidermis, dermis, and/or intramuscularly. The one or more neurotoxins may include botulinum toxin purified protein type A (“BTPP”) and/or the like. For example, botulinum toxin sold under Botox® or Dysport® branding may be a suitable neurotoxin.

The one or more neurotoxins may be measured in units. Units may be as determined by the company Allergan®, for example. The standard today is that Botox® is provided in a vial which contains exactly 100 Botox® units. A 100 Botox® unit vial is typically prepared for administration by adding 2.5 ml of one or more reconstitution sterile fluids which are liquid (100 units/2.5 ml). A 100 Botox® unit vial may be prepared for administration by adding up to 10 ml of one or more reconstitution sterile fluids (100 units/10 ml) or as little as 1 ml of one or more reconstitution sterile fluids (100 units/1 ml). A microneedle 30, 34 may include about 0.05 units or greater, about 0.10 unit or greater, about 0.20 units or greater, about 0.50 units or greater, or even about 1.00 unit or greater of the neurotoxin (e.g., BTPP). A microneedle 30, 34 may include about 100 units or less, about 70 units or less, about 50 units or less, about 40 units or less, about 30 units or less, or even about 10 units or less of the neurotoxin (e.g., BTPP). The quantity of neurotoxin may be adjusted based on target region (e.g., forehead, crow's feet), target depth (e.g., epidermis, dermis muscle), goals to be achieved (e.g., depth of wrinkle), or any combination thereof.

One or more microneedles 30, 34 may include one or more reconstitution sterile fluids that may function to reconstitute one or more neurotoxins, one or more dermal fillers, or both. The one or more reconstitution sterile fluids may function as a carrier. One or more carriers may cooperate with one or more other medications (e.g., medicament 21) to create a liquid suspension. One or more carriers may function to form the shape or structure of one or more frozen microneedles 30. One or more carriers may include any sterile liquid(s) suitable for reconstitution or dissolution. One or more carriers may include only saline. Saline may include preservative free 0.9% saline (i.e., contains sodium chloride 9.0 mg/ml and benzyl alcohol 9.0 mg/ml). Using only saline may allow for a more precise dissolution, avoid additional diluting of one or more medications (e.g., medicament 21), or both. One or more carriers may include saline in addition to one or more other carriers. One or more other carriers may include sterile water for injection (SWFI), dextrose 5% in water (D5W), Lactated Ringer's 5% dextrose in water (DSLRS), Ringers Solution, or any combination or concentration thereof. One or more carriers may include one or more other carriers without saline. The one or more microneedles 30, 34 may be free of any other carriers separate from the one or more reconstitution sterile fluids, stabilizing agents for the neurotoxin and or drug/medications/therapeutic agent (e.g., medicament 21) to be delivered.

The one or more carriers may be measured in liquid volume. A microneedle 30, 34 may include about 0.00125 ml or greater, about 0.00250 ml or greater, about 0.00500 ml or greater, about 0.01250 ml or greater, or even about 0.02500 ml or greater of the one or more carriers. A microneedle 30, 34 may include about 0.300 ml or less, about 0.250 ml or less, about 0.175 ml or less, about 0.125 ml or less, about 0.100 ml or less, about 0.075 ml or less, or even about 0.050 ml or less of the one or more carriers. The one or more carriers may be at a ratio to the one or more neurotoxins. The ratio may be about 0.05 ml or greater, about 0.10 ml or greater, about 0.25 ml or greater, or even about 0.50 ml or greater to 1 unit of the neurotoxin (i.e., 0.05 ml: 1 unit). The ratio may be about 3.0 ml or less, about 2.0 ml or less, about 1.5 ml or less, or even about 1.0 ml or less to 1 unit of the neurotoxin (i.e., 2.0 ml: 1 unit).

The one or more microneedles 30, 34 may be prepackaged with a lyophilized powder or quantified Botulinum toxin purified protein units and a corresponding amount (to yield a set dilution) of preservative free 0.9% or similar saline (at predetermined temperatures which preserves the integrity and function of the Botulinum toxin purified protein). The one or more microneedles 30, 34 may contain 100 BTPP units dissolved in 2.5 ml of preservative free 0.9% saline. A microneedle may contain between 2 units and 100 units of BTPP dissolved in between 0.25 ml or 0.05 ml of preservative free 0.9% saline, respectively.

One or more microneedles 30, 34 may include one or more dermal fillers. One or more dermal fillers may be useful in combination with or separate from the neurotoxin. The one or more dermal fillers may be provided in a same microneedle system (e.g., layered patches 20) as the neurotoxin (BTPP), a same microneedle as the neurotoxin, and/or even a separate microneedle system as the neurotoxin. The one or more dermal fillers may include hyaluronic acid (“HA”), (e.g., of various cross linking) (e.g., Juvederm Ultra® or Juvederm Voluma®), poly-L-lactic acid (“PLLA”), polyacrylamide, collagen (e.g., bovine collagen), polymethyl methacrylate (“PMMA”), crystals of hydroxyapatite or any combination thereof. Hyaluronic acid has been found to cooperate well with botulinum toxin for reducing side effects of BTPP injection and increase the lifespan of the HA; see Gauthier, N., “A Study That Highlights the Importance of Combining Botox and Hyaluronic Acid,” Dr. Nelly Gauthier Aesthetics Blog, https://www.docteurgauthier.fr/en/blog/combined-botox-hyaluronic-acid-treatment/, March 2023, pp. 1-3. The one or more dermal fillers may be measured in liquid volume. A microneedle 30, 34 may include about 0.025 ml, about 0.050 ml or greater, about 0.075 ml or greater, or even about 0.100 ml or greater of the one or more dermal fillers. A microneedle 30, 34 may include about 2.00 ml or less, about 1.75 ml or less, about 1.50 ml or less, about 1.25 ml or less, or even about 1.00 ml or less of the one or more dermal fillers. The ratio of dermal filler to one or more carriers may be about 1 mL of dermal filler to about 0.1 ml of one or more carriers (1 ml: 0.1 ml).

One or more microneedles 30, 34 may include one or more local anesthetics. The one or more local anesthetics may function to prevent and/or treat pain from the initial penetration and/or subsequent penetrations of one or more microneedles 30, 34. The one or more local anesthetics may allow for a patient to receive microneedle treatment more comfortably. One or more local areas may include lidocaine, articaine, tetracaine, mepivacaine, prilocaine, the like, or any combination thereof. One or more local people may include more specifically lidocaine 1%. The amount (e.g., volume) of local anesthetic may be chosen to be under toxic limits per application. For example, the total amount one or more microneedles 30, 34 (e.g., of a system 1) may have together of 1% lidocaine (plain, without vasoconstrictor) may be about 40 ml or less, about 20 ml or less, about 10 ml or less, about 5 ml or less, or even about 1 ml or less. The total number of the microneedles 30, 34 (e.g., of a system 1) may have together of 1% lidocaine (plain, without vasoconstrictor) may be 0.01 ml or more, 0.05 ml or more, 0.10 ml or more, 0.20 ml or more, or even 0.50 ml or more.

The system 1, 5 may include one or more arrays of microneedles 30, 34. Each substrate (e.g., patch 20) of a system 1, 5 may include one or more arrays thereon. The arrangement of arrays may allow for one or more microneedles 30, 34 to treat a localized area on a patient while each carrying a small amount of one or more medications (e.g., medicament 21). The arrays may be rows, columns, diagonal, the like, or any combination thereof. It is also possible that the one or more microneedles 30, 34 may be arranged on the substrate (e.g., patch 20) in non-array patterns, groups, randomized patterns, or a combination thereof. A substrate (e.g., patch 20) may include 1 or more, 2 or more, 4 or more, 5 or more, or even 10 or more microneedles 30, 34. A substrate (e.g., patch 20) may include 200 or less, 150 or less, 100 or less, or even 80 or less microneedles 30, 34.

The system 1, 5 may include a single layer or a plurality of layers of one or more microneedles 30, 34. The layers may include one or more frozen microneedles 30, one or more dissolvable polymeric microneedles 34, hollow, hydrogel forming, or a combination thereof. A plurality of layers may cooperate together to deliver one or more medications (e.g., medicament 21) (e.g., BTPP, HA, lidocaine) and to allow for self-administration of the one or more medications (e.g., medicament 21). A first layer of microneedles 30, 34 may penetrate the epidermis and stratum corneum while subsequent layers of microneedles 30, 34 may penetrate further. The layers may include one layer or more, two layers or more, or even three layers or more. The layers may include six layers or less, five layers or less, or even four layers or less. A first layer may include one or more leading microneedles 42. One or more subsequent layers may include one or more intermediate microneedles 44 and/or trailing microneedles 46. A plurality of layers may be comprised of nesting microneedles 40 or non-nesting microneedles 40. Nesting may mean that one microneedle rests partially within another microneedle 30, 34. Non-nesting may mean that while microneedles may be adjacent to and even stacked with one another, they are not nested (e.g., partially sitting inside another microneedle 30, 34).

A first layer of microneedles 30, 34 may include or be free of any BTPP, HA, or other ingredients. For example, the first layer may be free of any action causing medications (e.g., medicament 21) (e.g., BTPP, HA). The first layer may be mechanically the strongest to ensure successful penetration and formation of micropores and microchannels in the epidermis. Strength and mechanical ruggedness may be based on size, length, consistency, and geometry with respect to Young's modulus.

One or more subsequent layers of microneedles 30, 34 may be formed with less strength and/or mechanical ruggedness and focused on delivering medication. One or more subsequent layers of microneedles (MN) 30, 34 may carry one or more ingredients, BTPP, HA, one or more analgesics, local anesthetics and/or the like and even at different concentrations or viscosities. The one or more subsequent layers of microneedles 30, 34 may have longer length microneedles than the initial layer. Longer length microneedles may allow for microneedle shafts to retain a greater volume of medication and to penetrate to deeper skin or muscle levels. Advancement of each microneedle layer (such as after dissolution) may be achieved through direct manual pressure or by use of a tool (e.g., by hand, a roller, a compress, applicators etc.).

For example, a first layer of microneedles 30, 34 may have a length of 300 microns which is shorter than one or more subsequent layers of microneedles 30, 34. A first layer of microneedles 30, 34 may be formed of one or more dissolvable polymers which are stronger (mechanically) than one or more other dissolvable polymers forming one or more subsequent layers of microneedles 30, 34. A first layer of microneedles 30, 34 may have microneedles 30, 34 with tips 3 of a radius less than 10 microns, a base 4 of 300 microns, or both. One or more subsequent layers of microneedles 30, 34 may include medicated ingredients. One or more subsequent layers of microneedles 30, 34 may include one or more microneedles 30, 34 formed of one or more dissolvable polymers which have different viscosities, strength, rates of dissolution or both than the one or more dissolvable polymers of a first layer of microneedles 30, 34. One or more subsequent layers of microneedles 30, 34 may have a length of 500 microns or more and bases 4 of 2000 microns or less. One or more subsequent layers of microneedles 30, 34 may have shapes which are pyramidal, spiral, triangular conical, or a combination of those. One or more subsequent layers of microneedles 30, 34 may have tips 3 that can be sharp, spiral, pencil point, or other shapes.

The one or more layers of microneedles 30, 34 may include one or more nesting microneedles 40. Nesting microneedles 40 may be beneficial in providing microneedles with dedicated functions, creating micropores or microchannels for subsequent microneedles to enter into and further penetrate into skin or muscle, or both. Nesting microneedles 40 may allow for one microneedle to support a subsequent microneedle 30. Nesting microneedles 40 may allow for a much larger quantity (high density) of microneedles to be applied to a given area via the nesting arrangement than without the nesting. Nesting microneedles 40 may be more effective at piercing further (i.e., deeper) into the epidermis and/or dermis than the use of single layer microneedles. The multiple layers of microneedles may ensure 100% or near 100% penetration through the stratum corneum into the dermis, or even muscle, of medicated microneedles as resistance provided by the dermis and subcutaneous tissue to the microneedles is decreased after penetration of the stratum corneum barrier in the epidermis.

Previous studies have concluded that wider and longer microneedles can be more effective in skin penetration; see Davison, A., et al., “Transdermal drug delivery by coated microneedles: Geometry effects on effective skin thickness and drug permeability,” Chemical Engineering Research and Design, Vol. 86, Issue 11, November 2008, pp. 1196-1206, incorporated herein by reference in its entirety. Thus, in combining this finding with nested layers (e.g., nesting microneedles 40), progressively increasing the width and length of microneedles in subsequent layers can ensure staged advancement of the nested microneedles 40 into or through the epidermis, dermis, and/or muscle. As each microneedle 30, 34 only pierces a segment of the epidermis and/or dermis, an individual microneedle 30, 34 may be made shorter and more robust, thus reducing the likelihood of breaking and/or bending, while an overall combination of microneedles may offer a longer piercing depth than a single typical microneedle 30, 34 longer than 2000 microns even up to 5000 microns.

Nesting microneedles 40 may include one or more leading microneedles 42, one or more intermediate microneedles 44, one or more trailing microneedles 46, or any combination thereof. Each nesting microneedle 40 may include a hollow interior, a nesting cavity, or both. A hollow interior may function to hold one or more medications (e.g., medicament 21) therein. A nesting cavity may function to partially receive and/or retain a subsequent microneedle (e.g., tip 3 or part of microneedle 40). A nesting cavity may be formed at an end opposite an insertion end of a microneedle 40. A nesting cavity may be formed separate from a hollow interior or be one in the same. In other words, the nesting cavity may or may not be in fluid communication with the hollow interior. A nesting cavity may continue to the hollow interior. The nesting cavity may be separated from the hollow interior. Separation may be achieved by an interior wall. One or more microneedles 30, 34 may be formed longer, shorter, and/or equal in height and/or diameter (or other width) as one or more other microneedles 30, 34. One or more microneedles 30, 34 may have a length from about 50 microns to about 2,000 or more microns. It is also envisioned that layers of leading, intermediate, and trailing microneedles 42, 44, 46 may be non-nesting microneedles. In other words, the layers of microneedles may be adjacent to one another, stacked together, sequentially arranged, and/or the like, but not nesting at least partially within another microneedle 30, 34.

One or more leading microneedles 42 may function to pierce into skin, such as the epidermis. Even more specifically, a leading microneedle 42 may function to pierce and/or penetrate into or through the stratum corneum. A leading microneedle 42 may form a microchannel or micropore in the epidermis and/or dermis. This microchannel or micropore may allow for one or more subsequent microneedles to penetrate deeper, easier, and/or faster into the epidermis and/or dermis. A leading microneedle 42 may dissolve quickly at a predetermined known rate (to allow for subsequent microneedle(s) to penetrate (be manually advanced) into the created microchannel or micropore. A leading microneedle 42 may be a frozen microneedle 30, a dissolvable polymeric microneedle 34, or both. For example, a leading microneedle 42 may dissolve within 30 seconds to 10 minutes while a microchannel or micropore formed by a leading microneedle 42 may remain open for up to 24 hours. A leading microneedle 42 may deliver one or more medications (e.g., medicament 21) or be free of medication. One or more medications (e.g., medicament 21) may include any medication disclosed herein (e.g., neurotoxin, one or more stabilizing agents for the neurotoxin, reconstitution sterile fluids, one or more dermal fillers, one or more analgesics, local anesthetics, insulin, and/or anti-RNA antibodies, or the like). A leading microneedle 42 may be formed such as to be more robust (e.g., stronger) than subsequent microneedle(s) as a leading microneedle 42 functions to pierce through the stratum corneum of the epidermis. A leading microneedle 42 may have a height about equal to and/or shorter than one or more subsequent needles. A leading microneedle 42 may have a widest width about equal to and/or greater than one or more subsequent needles. A leading microneedle 42 may have a height sufficient to pierce into, and even through, the stratum corneum of an epidermis. A leading microneedle 42 may have a height of about 50 microns or greater, about 300 microns or greater, about 700 microns or greater, about 1,500 microns or greater, or even about 1,700 microns or greater. A leading microneedle 42 may have a nesting cavity, hollow interior, both, or neither. If a leading microneedle 42 is free of any medication, the leading microneedle 42 may include a nesting cavity while being free of a hollow interior. If a leading microneedle 42 includes one or more medications (e.g., medicament 21) (e.g., within the interior), the leading microneedle 42 may include both a nesting cavity and a hollow interior.

One or more intermediate microneedles 44 may function to deliver one or more medications (e.g., medicament 21) into the epidermis and/or dermis. An intermediate microneedle 44 may enter into the microchannel or micropore created by a leading microneedle 42. An intermediate microneedle 44 may pierce further into the epidermis and/or dermis (i.e., as compared to a leading microneedle). One or more intermediate microneedles 44 may include and/or be free of one or more medications (e.g., medicament 21). The one or more medications (e.g., medicament 21) may include any of the medications (e.g., medicament 21) discussed herein (e.g., one or more neurotoxins, one or more dermal fillers, one or more reconstitution sterile fluids, one or more analgesics or local anesthetics). One or more intermediate microneedles 44 may be formed of (e.g., if frozen) or include (e.g., if polymeric) one or more local anesthetics. For example, lidocaine, or more specifically lidocaine 1%. The one or more local anesthetics may function to prevent and/or treat pain from the initial microneedle penetration and/or subsequent penetrations. The one or more local areas may allow for a patient to receive microneedle treatment more comfortably. An intermediate microneedle 44 may include or be free of a nesting cavity, hollow interior, or both. For example, an intermediate microneedle 44 which is frozen and formed of the medication in frozen form, may have a nesting cavity but be free of a hollow interior. For example, an intermediate microneedle 44 which is a dissolvable polymeric microneedle 34 may have a nesting cavity and include a hollow interior in which one or more medications (e.g., medicament 21) may be provided. An intermediate microneedle 44 may have a height about less than, equal to, or greater than one or more leading microneedles 42, trailing microneedles 46, or both. An intermediate microneedle 44 may have a height and/or width sufficient to penetrate into the microchannel or micropore formed in the stratum corneum of an epidermis, pierce further into the epidermis, dermis and/or muscle, or a combination thereof. An intermediate microneedle 44 may have a height of about 100 microns or greater, about 300 microns or greater, about 700 microns or greater, about 1,500 microns or greater, about 1,700 microns or greater, or even about 2,000 microns or greater.

One or more trailing microneedles 46 may function to deliver one or more medications (e.g., medicament 21) into the epidermis and/or dermis. A trailing microneedle 46 may enter into the microchannel or micropore created by a leading microneedle 42, intermediate microneedle 44, or both. A trailing microneedle 46 may further pierce into the epidermis, dermis and/or muscle. A trailing microneedle 46 may include any of the one or more medications (e.g., medicament 21) listed herein (e.g., one or more neurotoxins, one or more dermal fillers). One or more trailing microneedles 46 may be formed of or include analgesic(s), local anesthetics, botulinum toxin type A, hyaluronic acid-based fillers, saline, the like or any combination thereof. A trailing microneedle 46 may be coated about its exterior with one or more analgesics, local anesthetics while botulinum toxin, hyaluronic acid, saline, and/or the like are located within and/or form the hollow shaft 48 and or walls of the microneedle 30. As a further example, a trailing microneedle 46 may be free of a local anesthetic or analgesic and only include one or more other medications (e.g., medicament 21) therein and/or forming the microneedle 30. A trailing microneedle 46 may be formed as a frozen microneedle 30. For example, a frozen microneedle as the trailing microneedle 46 may be free of a hollow interior and nesting cavity and may be formed from saline, BTPP, and/or HA. For example, a frozen microneedle 30 as a trailing microneedle 46 may include a hollow interior and be formed of saline and BTPP while having HA within the hollow interior or be formed of saline and HA while having BTPP (and optionally more saline) within the hollow interior. A trailing microneedle 46 may be formed as a dissolvable polymeric microneedle 34.

A dissolvable polymeric microneedle 34 may include a hollow interior. A hollow interior may include saline, BTPP, and/or HA therein. A trailing microneedle 46 may include one or more analgesics, local anesthetics within the hollow interior, forming or coating the exterior, or a combination thereof. A trailing microneedle 46 may have a height about less than, equal to, or greater than one or more leading microneedles 42, one or more intermediate microneedles 44, or both. A trailing microneedle 46 may have a height sufficient to penetrate into the microchannel or micropore formed in the stratum corneum of an epidermis and pierce further into the epidermis, dermis and/or muscle. A trailing microneedle 46 may have a height of about 100 microns or greater or even about 700 microns to about 1,500 microns or greater, about 1,700 microns or greater, or even about 2,000 microns or greater. One or more microneedles 30 may include a reservoir. A reservoir may be in fluid communication with a hollow shaft 48 of one or more microneedles 30, a surface of one or more substrates (e.g., patches 20), or both. A reservoir may function to refill one or more microneedles 30 with one or more medications (e.g., medicament 21), apply fluidic force to one or more microneedles 30 (propel botulinum toxin embedded microspheres of poli L amide upwards), or both. The reservoir may communicate with one or more microneedles 30 through a cavity in one or more substrates (e.g., patches 20), a cavity between a substrate (e.g., patch 20) and a tray 10, or both. A reservoir may be a syringe, a piston, IV bag pump, or other fluid container capable of supplying fluid under pressure. A reservoir may provide one or more medications (e.g., medicament 21) to the one or more microneedles 30. Pressure from one or more medications (e.g., medicament 21) may exert a mechanical force on one or more microneedles 30, one or more microspheres containing one or more medications (e.g., medicament 21), or both.

Pressure from one or more medications (e.g., medicament 21) may push one or more microneedles 30, one or more microspheres containing one or more medications (e.g., medicament 21), or both through or into the stratum corneum of a patient. A reservoir may provide pressure through one or more medications (e.g., medicament 21) which pushes one or more leading microneedles 42 through the stratum corneum of a patient, one or more intermediate or trailing microneedles 46 through a micropore or microchannel created by one or more leading microneedles 42, or both. A reservoir may provide pressure by compression of the reservoir, gravitational potential of the reservoir, both, or the like.

The material composition of a microneedle 30, 34 may vary. As discussed, one or more intermediate microneedles 44, one or more trailing microneedles 46, or both may function to deliver one or more medications (e.g., medicament 21) into the epidermis and/or dermis. Within an intermediate microneedle 44 and/or a trailing microneedle 46, frozen (−20° C. or other), preservative free 0.9% saline at a set volume, may be used as a carrier for the delivery of other medications (e.g., medicament 21). Along with the saline in an intermediate microneedle 44 or a trailing microneedle 46, lyophilized purified protein of botulinum toxin and/or hyaluronic acid can be found. The BTPP may also be embedded in the shaft of an intermediate microneedle 44 and/or a trailing microneedle 46 that comprises of hyaluronic acid and is hollow, or as coated tips or other variations. It is also possible hyaluronic acid dermal filler of various cross linking may be provided in the same microneedle system as the botulinum toxin or found in a separate microneedle system. Finally, one or more intermediate microneedles 44 and/or one or more trailing microneedles 46 may be formed of or include one or more local anesthetics such as lidocaine. The embodiments herein also describe polymeric (HA-hyaluronic acid) based microneedle trays 10 formed in a superimposed layered fashion. The layered microneedles may be formed as overlapping layers (three or more patches 20 superimposed on each other). Two to three or more layers of microneedles 30, 34 in peel off patches 20 may be applied to a desired area for treatment (e.g., forehead, glabella, periorbital region). A first layer of microneedles (e.g., one or more leading microneedles 42) may come into contact with the epidermis of the skin and penetrate into or through the stratum corneum. Upon penetration, the first layer of microneedles may form micropores and microchannels and then dissolve. After dissolution, one or more subsequent layers of microneedles (e.g., one or more intermediate microneedles 44, one or more trailing microneedles 46) (potentially medicated) may be able to penetrate the micropores and microchannels in the stratum corneum and further into the epidermis, dermis, and/or muscle. Layering of microneedles 30, 34 in overlapping layers (e.g., nesting) with size and shapes, and by selecting a suitable Young's modulus may solve limitations previously shown by polymeric microneedles 34, such as bending or breaking of the tips 3, incomplete penetration into the stratum corneum, medication spoilage at the epidermis, and even limited amount of medication administered through any given microneedle system. It also overcomes limitations of hollow microneedles and the potential for clogging of the needle openings with tissue during skin insertion; it also overcomes and solves the flow resistance due to dense dermal tissue compressed around microneedle tips 3 during insertion. Layered microneedles 30, 34 allow for administration of multiple (two or more) medications (e.g., medicament 21) with same cluster of overlapping patches 20, therefore allowing for the possibility of injected analgesics or local anesthetics through microneedles before administering highly viscous hyaluronic based filler or PLLA or Crystals of hydroxyapatite at a deeper dermal or subdermal level with subsequent microneedles 30, 34.

Other medical advantages besides correct dilution and ease of use of the embodiments herein are: saved time spent by medical professionals (no need to dilute BTPP powder and reduced costs), saved travel time to a professional's office by a patient, no need for trained medical assistants in the office setting or booking appointments (telemedicine appointments can be directly booked in a doctor's online calendar) resulting in better revenue for the medical professional, lower costs for patients, and a standardized price for the cosmetic application of BTPP. Patient loyalty is preserved for each medical office as the patient traveling or relocating doesn't necessarily require a switch in the medical professional because self-application under controlled remote medical supervision is a breakthrough in BTPP administration. This breakthrough can be extended to other medications (e.g., medicament 21) which were previously not self-administrable. Given the sterile preparation of the product(s) and sterile and safe guided administration, the trauma, infection and bruising associated with BTPP administration while using hypodermic needles is reduced or entirely eliminated. One of the cardinal advantages is also the mitigation of pain when compared to that of a traditional hypodermic needle injection.

The system 1, 5 may relate to a self-administered microneedle patch 20. The self-administered microneedle patch 20 may simply be referred to as a patch 20 or a microneedle patch 20. A microneedle patch 20 with one or more microneedles 30 carrying BTPP (and even additional medications) therein may be referred to as a BTPP patch 20. A patch 20 may function to easily solve the challenges of traditional dermal or even intramuscular medication administration by providing for safe self-administration under remote (telemedicine) medical guidance. For example, the patch 20 may include a substrate carrying one or more layers and/or arrays of microneedles 30, 34 thereon. The substrate may be a patch 20, patch like, part of a patch 20, supported by a patch 20, or any combination thereof. The patch 20 may be a removable adhesive patch 20 or otherwise self-adhering patch 20. The one or more microneedles 30, 34 thereon may be one or more frozen microneedles 30 and/or one or more dissolvable polymeric microneedles 34. For example, the substrate (e.g., patch 20) may have thereon frozen 0.9% or similar preservative free saline microneedles (1-12 or even more microneedles 30, 34 per tray 10) filled with quantitative (2-10 or more) units of BTPP per microneedle 30, 34.

The system 1, 5 may include one or more substrates (e.g., patches 20). The one or more substrates (e.g., patches 20) may each function to be temporarily applied onto an epidermis of a patient, carry one or more microneedles 30, 34, or both. The system 1, 5 may include one or more substrates (e.g., patches 20) removably adjoined to one or more other substrates (e.g., patches 20). One or more substrates (e.g., patches 20) may be removably adjoined to one or more other substrates (e.g., patches 20) via one or more scoring lines, perforations 22, and/or the like. A plurality of substrates (e.g., patches 20) may be formed in rows, columns, or both. For example, the system 1, 5 may include a single row of 2 to 10 substrates (e.g., patches 20). As another example, the system 1, 5 may include 2 to 10 rows (e.g., adjacent rows) and 2-10 columns (substrates (e.g., patches 20) per row).

The one or more substrates (e.g., patches 20) may be formed of one or more materials suitable for the one or more microneedles 30, 34 as discussed above or may differ. The one or more substrates (e.g., patches 20) may function to adhere or otherwise support one or more microneedles 30, 34 for insertion into a patient's skin, allow for pressure to be applied to one or more microneedles 30, 34, or both. The one or more substrates (e.g., patches 20) may or may not be adherable to a patient's skin. The one or more substrates (e.g., patches 20) may be formed as dissolvable. Dissolvable may mean that the one or more substrates (e.g., patches 20) may be formed of one or more materials which are highly water soluble. Dissolvable may mean that the temperature of the receiving skin may melt or otherwise dissolve the one or more substrates (e.g., patches 20). Dissolvability may be achieved via freezing, being formed of a polymer, or both. Dissolvability may be beneficial in eliminating biohazardous waste. The one or more substrates (e.g., patches 20) may be formed as one or more adhesive patches 20. An adhesive patch 20 suitable for medical use may be made by any material suitably known. It is also contemplated that the substrate (e.g., patch 20) may just be a carrier of one or more microneedles 30, 34 (e.g., non-adhesive) while a separate adhesive patch 20 supports the substrate (e.g., patch 20) or is located about the substrate (e.g., patch 20) for adhering the system to a patient's skin (e.g., akin to an adhesive bandage). Such a separate adhesive patch 20 may be referred to as a tray 10.

The system 1, 5 may include or be free of a single tray 10 or a plurality of trays 10. The tray 10 may function to support one or more substrates (e.g., patches 20), one or more microneedles 30, or both. The tray 10 may function to adhere the system to a patient's skin. A tray 10 may function like an adhesive portion of a bandage. A tray 10 may be formed as an adhesive patch 20. The tray 10 may be made of any medically suitable material for temporarily adhering to a patient's skin. The tray 10 may be bonded to one or more substrates (e.g., patches 20) via the same adhesive used for adhesion to skin or using any other suitable means. The tray 10 may include or be free of one or more scoring lines, perforations 22, and/or the like. The scoring lines, perforations 22, and/or the like may be aligned with those of a substrate (e.g., patch 20). A single tray 10 may carry a single or a plurality of substrates (e.g., patches 20) thereon. The tray 10 may have an overall area larger than that of a substrate (e.g., length and width). The tray 10 may extend beyond the substrate (e.g., patch 20). This may allow for an adhesive of the tray 10 to be exposed and adhere to the skin of a user.

The system 1, 5 may include or be free of a single removable cover or a plurality of removable covers. A plurality of removable covers may function to carry one or more substrates (e.g., patches 20), trays 10, or both thereon, serve as packaging, protect an adhesive, or any combination thereof. A removable cover may be reusable. A removable cover may be any suitable material for temporarily adhering to a substrate and/or tray 10.

The embodiments herein provide for a method of self-administration and prepackaging of BTPP and/or HA into frozen and/or polymeric microneedle trays 10 with adhesive peel off patches 20. The embodiments herein provide for an improved shape of one or more dissolvable polymeric microneedles 34, one or more frozen microneedles 30, or both which may be medicated for deeper skin penetration (e.g., spiral but other shapes can be considered as well). Two to three or even more different sizes and shapes (pencil point, spiral, quincke, atraucan, sprotte-like) of microneedles 30, 34, incorporating saline, BTPP, and/or HA can be grouped in a self-adhesive patch 20 to be peeled of and applied to one targeted skin area (e.g., over the corrugator muscle). This may allow different skin penetration depths and dissolution areas of one or more medications (e.g., medicament 21), avoiding potential side effects. For example, a shorter leading microneedle 42 targeting the corrugator supercilis muscle is nested or paired with a longer intermediate microneedle 44 or trailing microneedle 46 that will deliver BTPP at the procerus level or vice-versa.

For example, a patient will press the microneedle(s) 30, 34 by applying pressure on the patch 20. Depending on the shape of the microneedle 30, 34 the needle may rotate when pressed (e.g., spiral microneedles 32). Additionally, a roller (e.g., face roller) can be used on top of the patch 20 and rolled a few times back and forth to enhance absorption and facilitate deeper penetration. The microneedles patch 20 can be applied for a certain amount of time to allow absorption through the skin. For example, the microneedles 30, 34 can be applied for 15 to 30 minutes, but longer applications may be considered. The materials and shape of the BTPP containing microneedles 30, 34 may provide for easier penetration through the skin and allow for the entire microneedle 30, 34 to be delivered within the targeted organ (dermis or muscle), not just the tip 3 of the microneedle 30, 34. Especially when used in multiple layers (e.g., two to three or more) of adherent patches 20 with microneedle trays 10, the medicated (BTPP and/or HA) microneedles 30, 34 of the subsequent layers in a patch 20 may penetrate the skin easier, with less mechanical resistance, and deeper for targeted delivery in the dermis or even intramuscular delivery.

Another embodiment provides a method 200 of applying the medicine of the system 1 of any of the above. The method 200 comprises (a) removing one or more adhesive patches 20 from a tray 10; (b) applying the one or more adhesive patches 20 to an epidermis of the patient such that the one or more frozen microneedles 30 penetrate through an epidermis to a dermis level or deeper; and (c) the one or more frozen microneedles 30 melting at the patient's skin temperature and delivering the medicine to the target site on the patient within the dermis or further anatomical layers. The method 200 may further comprise (a) prewarming skin temperature of a desired anatomical site to apply the medication and/or the adhesive patches 20 with an infrared light, a warm compress, and/or other suitable methods; (b) optionally, pretreating the area of skin with salicylic acid or alike chemical peel to disrupt cellular junctions in the epidermis and facilitate penetration of the microneedle; (c) removing one or more adhesive patches 20 from a tray 10; (d) applying the one or more adhesive patches 20 to the skin of the patient such that the one or more frozen microneedles 30 from the tray 10 penetrate through an epidermis to a dermis level or deeper; and (e) the one or more frozen microneedles 30 melting at the patient's skin temperature and delivering the composite medicine to the target site within the dermis or further anatomical layers.

The method 200 may further comprise (a) a plurality of nesting microneedles 40 in overlapping patches 20 applied to the skin; (b) applying constant pressure to the nesting microneedles 40 such that a first layer of leading microneedles 42 penetrates into the stratum corneum, dissolves, and forms a plurality of microchannels; (c) continuing to apply pressure to the nesting microneedles such that one or more subsequent layers of intermediate microneedles 44 and/or trailing microneedles 46 advance through the microchannels and into the dermis or even deeper (e.g., intramuscular); and (d) optionally, reapplication of additional nesting microneedles (e.g., additional patches 20) at a later time (e.g., 3 months) after dissolution.

The embodiments provide a self-administered prefilled BTPP tray 10 (may be referred to as BTPP tray 10 or tray 10 hereinafter). The BTPP tray 10 may function to solve the challenges of traditional BTPP administration by providing for safe administration under remote (telemedicine) medical guidance of patch like (peel off) frozen 0.9% or similar preservative free saline microneedle trays 10 (1-12 or even more microneedles 30 per tray 10) filled with quantitative (2-10) units per microneedle. The BTPP trays 10 may include a tray 10 having one or more patches 20 and one or more microneedles 30. The tray 10 may allow for easy packaging, transportation, and storage of the medicine within the microneedles 30. The microneedles 30 may be provided as frozen microneedles 30. The frozen microneedles 30 may be frozen composite saline/BTPP microneedles 30.

The BTPP tray 10 may include one or more frozen microneedles 30. The frozen microneedles 30 may function to provide the BTPP in the correct concentration, allow for insertion into the skin of a patient, or both. Replacing the vials with frozen (at temperatures that allow preservation of the BTPP) saline microneedle shaped trays 10 prevents the need and related dilution errors by medical professionals and delivers accurate amount of botulinum toxin purified protein units per site of dermal/muscle penetration.

Each microneedle may include saline. The amount of preservative free saline used to build each of the microneedle to harbor e.g., 5 units of BTPP (the concentration can vary per microneedle and or tray 10 depending on the target tissue and clinical applications) can and will be quantified to yield the right dilution (e.g., 0.5 ml). The frozen saline (shaped into a microneedle) containing lyophilized BTPP mixes and dissolves at a target level, intradermal or intramuscular after skin penetration, each specific tray 10 size being designed for a specific target (intradermal, subdermal, intramuscular).

Each microneedle may be formed as an ice microneedle. The ice microneedles 30 may be made from water, GelMA, Alg, and Matrigel® solutions. It is contemplated that each microneedle contains frozen (−20° C. or other), preservative free 0.9% saline at a set volume (for example, from 0.5 ml) and lyophilized purified protein of botulinum toxin set units (from two or more).

Instead of using a carrier as described in U.S. Pat. No. 8,420,105, the embodiments herein may provide a composite of preservative free 0.9% saline (frozen) and lyophilized BTPP powder (to be released and mixed up at target level), no additional water or soluble materials are used as carriers that could dilute further the concentration (upon dissolution and therefore changing absorption at the target level). The BTPP may also be embedded into a shaft 48 of a hollow hyaluronic acid needle or possibly as coated tips or other variations.

The microneedle may include a dermal filler-hyaluronic acid of various cross linking. The hyaluronic acid may be beneficial in combination with botulinum toxin, help reduce side effects due to the needle piercing, or both. Side effects may include bruises, intraarterial injection, and even nodules. The hyaluronic acid may be provided in the same microneedle system (layered patches 20) as the botulinum toxin or a separate microneedle system.

The microneedle may come in one or more shapes. The one or more shapes may be a spiral or triangular or another shape that allows complete skin penetration with minimal force required. For example, a microneedle can be thinner at the tip 3 and wider/thicker at the base 4 of the tray 10/peel off patch. Such shape may allow entire dissolution of material at the desired skin depth by hand application. For example, the patient will press and rotate the (spiral) microneedle(s) patch 20 through the epidermis of the skin. Additionally, a roller can be used on top of the patch 20 a few times back and forth to enhance absorption and deeper penetration. The microneedles patch 20 can be applied for a certain amount of time to allow absorption through the skin (suggested 15 to 30 min). The materials and shape of the BTPP containing frozen microneedles 30 may provide for easier penetration through the skin and allow entire material to be mixed up and delivered within the targeted organ (dermis or muscle) not just the tip 3. Especially when used in multiple layers (e.g., 2 or more) of adherent patches 20 with microneedle trays 10, the medicated (BTPP and/or HA) microneedles 30 of the subsequent layers in a patch 20 may penetrate the skin easier, with less mechanical resistance, deeper for targeted delivery in the dermis or even intramuscular delivery.

The microneedle 30 may have a shape which provides for the frozen medicated composite to have deeper skin penetration. The shape may be spiral but other shapes can be considered as well. Other shapes may include pencil point, spiral, quincke, atraucan, sprotte, the like, or any combination thereof. The needles may be provided in two to three or more different sizes and shapes on a single patch 20 or tray 10.

The frozen microneedles 30, incorporating saline and BTPP and/or lidocaine or HA can be grouped in a self-adhesive patch 20 to be peeled off and applied to one targeted skin area (e.g., corrugator muscle). This allows different skin penetration and dissolution of the material, avoiding potential side effects (for example a shorter microneedle targeting the corrugator supercilis muscle while a longer microneedle will deliver BTPP at procerus level or vice-versa).

The microneedles 30 may be separated from each other into the tray 10 with the possibility of individual application of each microneedle, or a group of microneedles 30, not the entire tray 10 altogether. The microneedles 30 are not only advantageous in their material and shape, but they can also be delivered intramuscular (for cosmetic and pain management or even other applications), not only intradermal.

The embodiments herein may provide for a method 200 of using the BTPP trays 10 and microneedle(s). The microneedles 30 may be provided to a patient via a BTPP tray 10. Based on the amount desired, such as by a prescription or guidance of medical professional, the number of desired microneedles 30 or patches 20 may be peeled off of the BTPP tray 10. Prior to peeling and/or application, a patient may first alcohol prep their skin (e.g., sanitize) where the patch 20 and/or microneedles 30 may be placed. A patient may apply and insert one or more microneedles 30 by rotating through the epidermis at the target level. The depth of the microneedle tray 10 and volume will determine the penetration depth and amount of BTPP units delivered.

The microneedles patches 20 and/or trays 10 may include one or more nesting microneedles 40. Nesting microneedles 40 may be used for providing microneedles 30 with dedicated functions. Nesting microneedles 40 may be more effective at piercing further (i.e., deeper) into the epidermis and/or dermis than the use of a single layer microneedle 30. The multiple layers of microneedles 40 in overlapping patches 20 may ensure 100% penetration through the stratum corneum into the dermis or even intramuscular of medicated microneedles 40 as resistance provided by the dermis and subcutaneous tissue to the microneedles 40 is decreased after penetration of the stratum corneum barrier in the epidermis. Wider and longer microneedles 40 can be more effective in skin penetration. Therefore, progressively increasing the width and length of microneedles 40 in subsequent layers can ensure advancement of a third microneedles tray 10 through a second microneedles tray 10 through a first microneedles tray 10 into the epidermis. As each microneedle 40 only pierces a segment of the epidermis and/or dermis, the microneedles 40 may be made shorter and more robust, thus reducing the likelihood of breaking and/or bending. Nesting microneedles 40 may include one or more leading microneedles 42, intermediate microneedles 44, trailing microneedles 46, or any combination thereof. Each nesting microneedle 40 may include a hollow interior, a nesting cavity, or both. A hollow interior may function to hold one or more medicaments 21 therein. A nesting cavity may function to receive and/or retain a subsequent microneedle 40 (tip 3 or part of microneedles). One or more microneedles 40 may be formed longer, shorter, and/or equal height and/or diameter as one or more other microneedles 40. Moreover, one or more microneedles 40 may have a length from about 50 microns to about 2000 or more microns.

One or more leading microneedles 40 may function to pierce into the skin, such as the epidermis. Even more specifically, a leading microneedle 42 may function to pierce and/or penetrate into the stratum corneum. The leading microneedle 42 may form a microchannel in the epidermis and/or dermis. This microchannel may allow for one or more subsequent microneedles 40 to penetrate easier and faster into the epidermis and/or dermis. The leading microneedle 42, being frozen, may dissolve quickly to allow for the subsequent needles 40 to penetrate into the microchannel. For example, a leading microneedle 42 may dissolve within 30 seconds to 10 minutes while a microchannel formed by the leading microneedle 42 may remain open for up to 24 hours. The leading microneedle 42 may deliver one or more medications or be free of medication. The leading microneedle 42 may be formed such as to be more robust (e.g., stronger) than subsequent microneedles 40 as the leading microneedle 42 functions to pierce through the stratum corneum. The leading microneedle 42 may have a height about equal to and/or shorter than one or more subsequent needles. The leading microneedle 42 may have a height sufficient to pierce into, and even through, the stratum corneum of an epidermis. The leading microneedle 42 may have a height of about 50 microns or even about 300 microns to about 700 microns to even about 1,500 microns.

One or more intermediate microneedles 44 may function to deliver one or more medicaments 21 into the epidermis and/or dermis. The intermediate microneedle 44 may enter into the channel created by a leading microneedle. The intermediate microneedle 44 may further piece into the epidermis and/or dermis. One or more intermediate microneedles 44 may be formed of or include one or more anesthetics; for example, lidocaine such as lidocaine 1%. The anesthetic(s) may function to prevent and/or treat pain from the initial penetration and/or subsequent penetrations. The one or more anesthetics may allow for a patient to more comfortably receive the micro needling treatment. The intermediate microneedle 44 may have a height about less than, equal to, or greater than one or more leading microneedles 42, trailing microneedles 46, or both. The intermediate microneedle 44 may have a height sufficient to penetrate into the channel formed in the stratum corneum of an epidermis, pierce further into the epidermis and/or dermis. The intermediate microneedle 44 may have a height of about 300 microns or greater or even 700 microns to about 1,500 microns or greater, about 1,700 microns or greater, or even about 2,000 microns or greater.

Furthermore, one or more trailing microneedles 46 may function to deliver one or more medicaments 21 into the epidermis and/or dermis. The trailing microneedle 46 may enter into the channel created by a leading microneedle 42, intermediate microneedle 44, or both. The leading microneedle 42 may further pierce into the epidermis and/or dermis. One or more trailing needles 46 may be formed of or include one of anesthetics, botulinum toxin, hyaluronic acid-based fillers, saline, the like or any combination thereof. For example, the trailing microneedle 46 may be formed as a hollow ice needle as disclosed hereinbefore which is further coated about its exterior with an anesthetic while botulinum toxin, hyaluronic acid, saline, and/or the like are located within the hollow shaft. As a further example, the trailing microneedle 46 may be free of an anesthetic and only include medicament 21 therein. The trailing microneedle 46 may have a height about less than, equal to, or greater than one or more leading needles, intermediate needles, or both. The trailing microneedle 46 may have a height sufficient to penetrate into the channel formed in the stratum corneum of an epidermis, pierce further into the epidermis and/or dermis. The trailing microneedle 46 may have a height of about 100 microns or greater or even about 700 microns to about 1,500 microns or greater, about 1,700 microns or greater, or even about 2,000 microns or greater.

The embodiments herein may provide for a method 200 of using the BTPP trays 10 and microneedle(s). The microneedles may be provided to a patient via a BTPP tray 10. Based on the amount desired, such as by a prescription or guidance of medical professional, the number of desired microneedles or patches 20 (layered) may be peeled off of the BTPP tray 10. Prior to peeling and/or application, a patient may first alcohol prep their skin (e.g., sanitize) where the patch 20 and/or microneedles may be placed. A patient may apply and insert one or more microneedles by rotating through the epidermis at the target level. The number of layers and depth of the microneedle tray 10 and volume will determine the penetration depth and amount of BTPP units delivered.

The microneedles 30 provided by the embodiments herein may be manufactured in a way that uses the most efficient tools and materials. Exemplary methods and materials are discussed in Larraneta, E., et al., “Microneedle arrays as transdermal and intradermal drug delivery systems: Materials science, manufacture and commercial development,” Materials Science and Engineering R: Reports, Volume 104, Apr. 13, 2016, pp. 1-32, which is incorporated herein by reference in its entirety. Another suitable method for preparing frozen microneedles 30, such as with freeze dry technology, can be found in Chinese Patent Application No. 115089864A, incorporated herein by reference in its entirety.

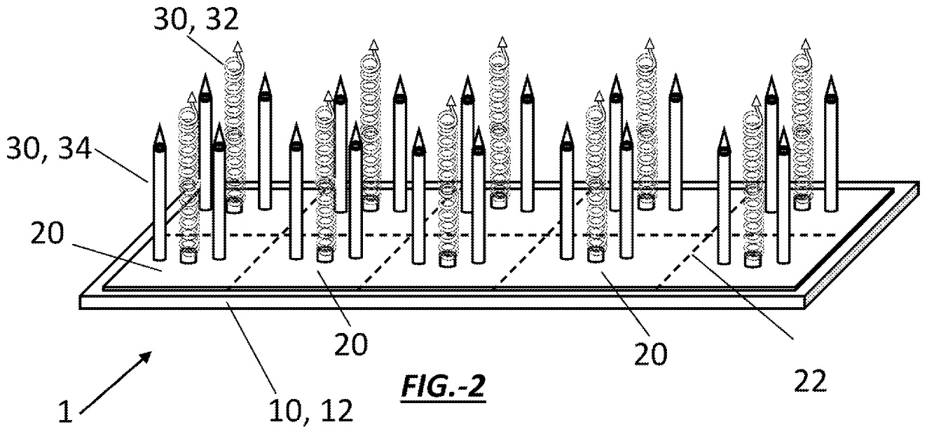

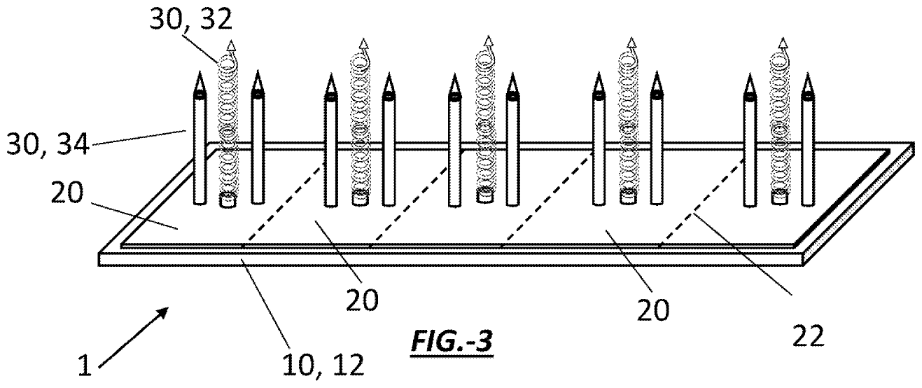

FIGS. 1 to 3 illustrate a system 1 according to an embodiment herein. The system 1 includes a tray 10. The tray 10 may be a self-adhesive tray 12, in an example. Adhered to the tray 10, 12 is a plurality of adhesive patches 20. Located on each adhesive patch 20 is a plurality of frozen microneedles 30. The frozen microneedles 30 are displayed in a variety of sizes and shapes. One or more of the microneedles 30 is illustrated as a spiral frozen microneedle 32. Each adhesive patch 30 includes one of the spiral frozen microneedles 32. Herein, microneedles 30 and microneedles 32 may be used/described interchangeably. Other frozen microneedles 30 are illustrated as pencil-shaped microneedles in the figures. The frozen microneedles 30 are shown grouped as arrays, clusters, symmetrical, asymmetrical, patterned, and random groupings on each adhesive patch 20. The plurality of adhesive patches 20 are shown as dividable by perforations 22 (exemplary means of maintaining patch together or separating one patch from others). FIG. 2, with reference to FIG. 1, illustrates the frozen microneedles 30 in two linear arrays according to an example. FIG. 3, with reference to FIGS. 1 and 2, illustrates the frozen microneedles 30 in a single linear array according to an example.

The embodiments herein may further comprise any one or more of the features described herein in any combination, including the preferences and examples listed in this specification, and including the following features: a predetermined reconstitution of saline and BTPP per microneedle 30, 34, a predetermined depth of penetration, most of the microneedle 30, 34 as opposed to just the tip 3 may be dissolved in the target tissue, and the ability to provide for individual microneedle 30, 34 insertion or a patch line or even a grouping as prescribed.

There can be a variety of shapes and sizes microneedles 30 of different numbers e.g. 2-3 to 10-12 microneedles per tray 10. This may allow for the user to exercise slight pressure manually to the patch 20 of microneedles 30 on the skin or apply pressure through rotating actions to ensure penetration of such frozen microneedles 30 through the skin to deliver corresponding amount of BTPP in the preservative free saline of the frozen microneedle 30 now reaching a fluid state at targeted subdermal level temperatures (e.g., body temperature).

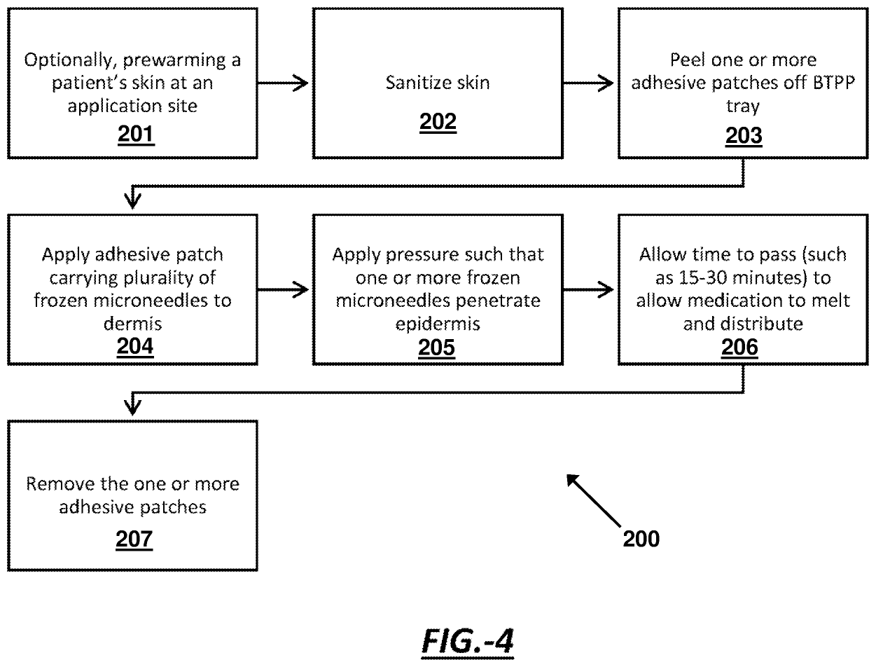

In FIG. 4, with reference to FIGS. 1 through 3, a flowchart depicts a method 200 of using the tray 10 according to an example. As shown in block 201, prior to application, the application site of the patient may be warmed. The warming may allow for the microneedle 30, 34 to more quickly melt and be delivered into the dermis or further anatomical layers. As shown in block 202, prior to application, the application site will be sanitized. As shown in block 203, during use, the frozen microneedles 30 are peeled off in as many numbers as desired (2-12 or even more) from the removable cover and then, as indicated in block 204, the adhesive patch 20 is applied on the skin. As shown in block 205, pressure is applied such that one or more frozen microneedles 30 penetrate the epidermis. As shown in block 206, the frozen microneedles 30 are allowed to stay in contact with the skin for 15-30 minutes until the microneedles 30 have pierced the epidermis; hand pressure will completely immerse microneedles 30 at subdermal level for reaching a fluid mixed compound state between the saline and the botulinum toxin purified protein. A hand roller or applicator can be applied up and down a few times (e.g., rolled over the patch 20) with soft and/or constant pressure on the microneedles 30 to ensure complete skin penetration. As shown in block 207, the one or more adhesive patches 20 are removed.

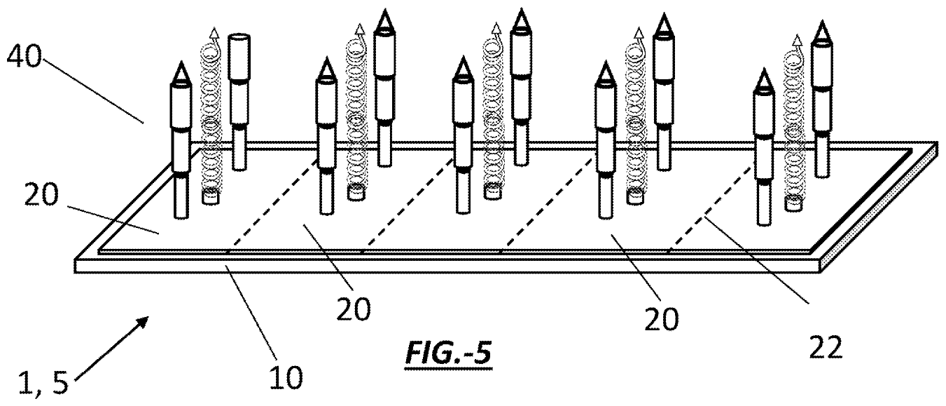

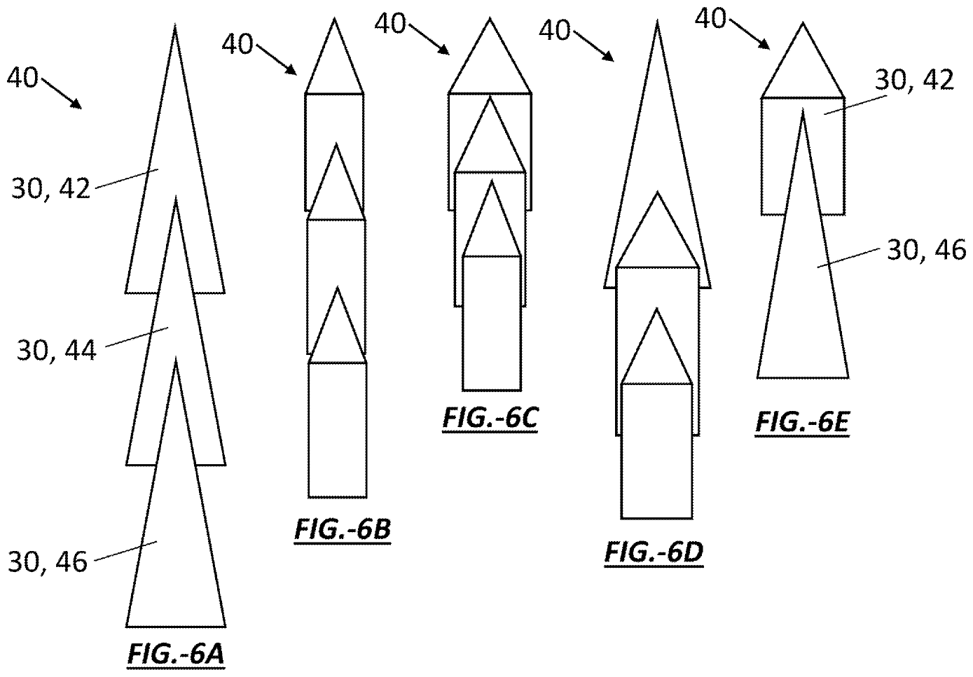

FIG. 5, with reference to FIGS. 1 through 4, illustrates a system 1 which is embodied as a multilayer system 5 according to another example. The system 1, 5 includes a tray 10. Adhered to the tray 10 may be a plurality of adhesive patches 20. The adhesive patches 20 may be dividable by perforations 22. Located on the substrate (e.g., patches 20) may be a plurality of frozen microneedles 30. The frozen microneedles 30 included a plurality of nested microneedles 40. FIGS. 6A-6E, with reference to FIGS. 1 through 5, illustrate examples of nested microneedles 40. The nested microneedles 40 may include multiple layers of frozen microneedles 30. The nested microneedles 40 may include a leading microneedle 42. Nested within the leading microneedle 42 may be an intermediate microneedle 44 or a trailing microneedle 46.

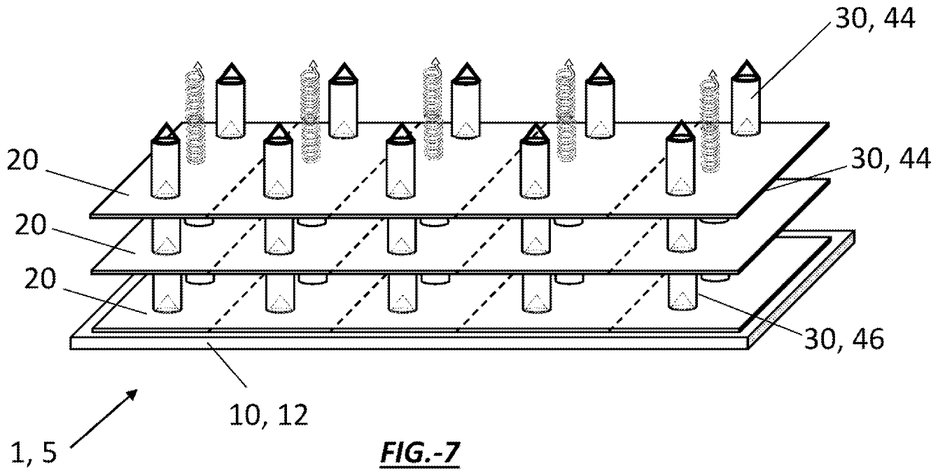

FIG. 7, with reference to FIGS. 1 through 6E, illustrates a system 1 which is embodied as a multilayer system 5 according to another example. The system 1, 5 includes a tray 10. Adhered to the tray 10 are a plurality of adhesive patches 20. The adhesive patches 20 all have a plurality of microneedles 30 thereon. The patches 20 include leading microneedles 42, intermediate microneedles 44, and trailing microneedles 46.

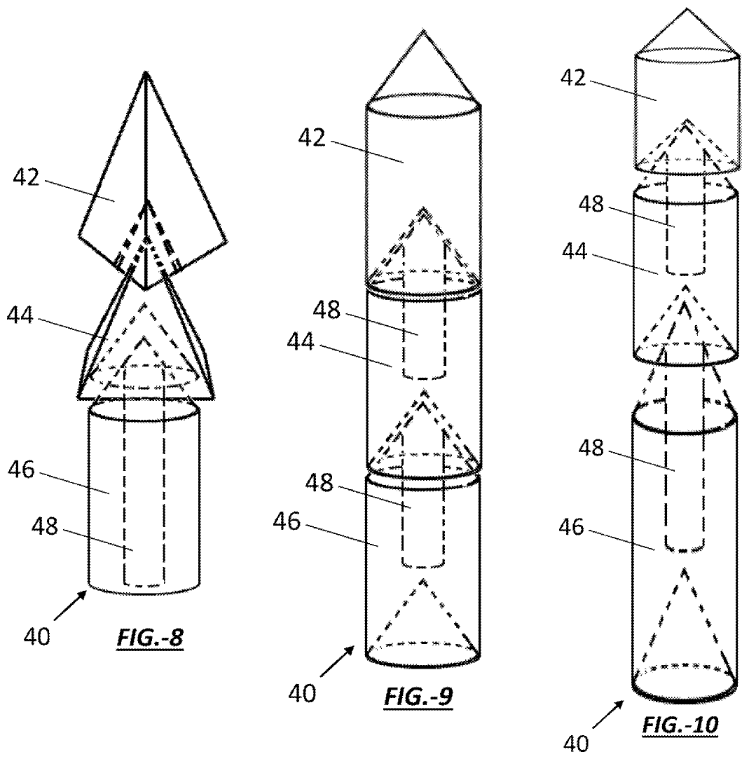

FIG. 8, with reference to FIGS. 1 through 7, illustrates nested microneedles 40 according to another example, in which the leading microneedle 42 is a triangular prism shape, the intermediate microneedle 44 is a pyramidical shape, and the trailing microneedle 46 has a conical shape with a hollow shaft 48 carrying medication therein (e.g., BTPP, HA). FIG. 9, with reference to FIGS. 1 through 8, illustrates nested microneedles 40 according to another example. The leading microneedle 42 may be a polymeric HA microneedle. The intermediate microneedle 44 includes a hollow shaft 48 carrying medication therein, such as BTPP. The trailing microneedle 46 includes a hollow shaft 48 carrying medication therein such as HA. The HA in the trailing microneedle 46 may have a higher viscosity.

FIG. 10, with reference to FIGS. 1 through 9, illustrates nested microneedles 40 according to another example. The microneedles 40 have a height which progressively increases. For example, the leading microneedle 42 may be a 400 polymeric HA microneedle, the intermediate microneedle 44 may be a 700 HA or BTPP frozen microneedle 30, and the trailing microneedle 46 may be a 1700 HA or BTPP frozen microneedle 30.

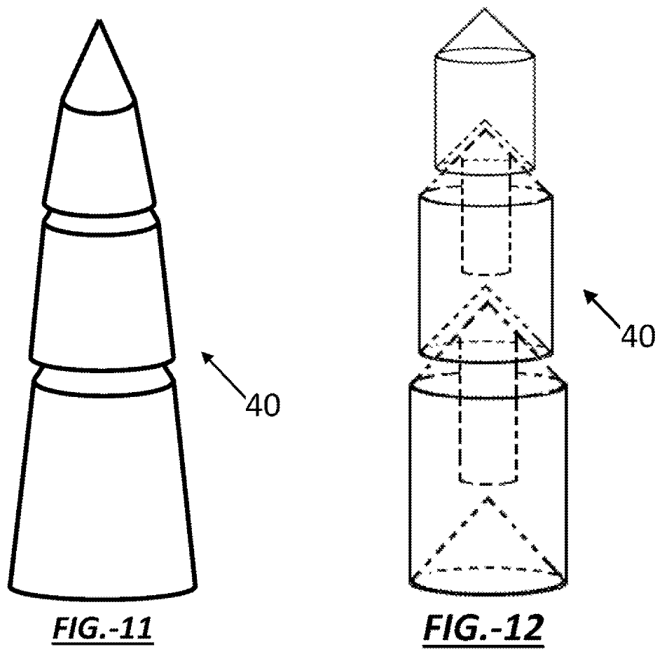

FIG. 11, with reference to FIGS. 1 through 10, illustrates nested microneedles 40 according to another example. The microneedles 40 have an increasing height from the leading microneedle 42 to the trailing microneedle 46. Each microneedle 40 also has an individually increasing width (e.g., tapered). The nested microneedles 40 also progressively increase in width as a whole along the entire system's height (e.g., the leading microneedle 42 has a smaller width than the trailing microneedle 46).

FIG. 12, with reference to FIGS. 1 through 11, illustrates nested microneedles 40 according to another example. The microneedles 40 have an increasing height from the leading microneedle 42 to the trailing microneedle 46. The nested microneedles 40 also progressively increase in width as a whole along the entire system's height (e.g., the leading microneedle 42 has a smaller width than the trailing microneedle 46).

The embodiments herein provide a system 1 and method 200 for delivering a medicament 21 in liquid suspension (such as Botulinum toxin purified protein (“BTPP”) and a preservative free saline). The system 1 includes: (a) one or more frozen microneedles 30 formed of the liquid suspension in frozen form and configured for insertion into a dermis (via insertion through the epidermis) of a patient and then subsequent melting and delivery into the dermis or even further layers of the patient; (b) one or more adhesive patches 20 configured to be temporarily applied onto the epidermis of the patient, wherein the adhesive patch 20 carries the one or more frozen microneedles 30 thereon; and (c) a tray 10 which is removably adhered to the adhesive patch 20 on an opposite side as the one or more frozen microneedles 30.

Additionally, several aspects of the embodiments herein are further set out in the following numbered clauses:

Clause 1. A system 1 for delivering one or more medications 21 through an epidermis of a patient, wherein the system 1 includes: a) one or more microneedles 30 which are dissolvable and configured for insertion into a dermis via insertion through an epidermis of the patient's skin and delivery of the one or more medications 21, drugs, active ingredients into the dermis and/or even further layers of the skin, wherein the one or more microneedles 30 include: (i) one or more neurotoxins or recombinant neurotoxin and/or one or more dermal fillers; (ii) one or more carriers; (iii) optionally, one or more analgesics or local anesthetics; b) one or more substrates (e.g., patches 20) configured to be temporarily applied onto the epidermis of the patient, wherein the one or more substrates carry the plurality of microneedles thereon; c) optionally, one or more trays 10 affixed to the one or more substrates on an opposite side as the one or more microneedles 30, wherein the one or more trays 10 include the one or more adhesive patches 20; and d) optionally, one or more removable covers which are removably adhered to the one or more substrates and/or one or more trays 10.

Clause 2. The system 1 of Clause 1, wherein the one or more microneedles 30 are in the form of one or more frozen microneedles 30, one or more polymeric microneedles 34, or both.

Clause 3. The system 1 of Clause 1 or 2, wherein the one or more neurotoxins are included in the one or more microneedles 30 as coated tips 65, lyophilized powder laser engraved in the base 4 or embedded in micro spheres of poli L amide or HAx-within the hollow cavity and wherein the one or more neurotoxins include a botulinum toxin purified protein type A.

Clause 4. The system 1 of any of the preceding Clauses, wherein the one or more carriers is a sterile saline solution.

Clause 5. The system 1 of Clause 4, wherein the saline solution is preservative free 0.9% saline.

Clause 6. The system 1 of any of the preceding Clauses, wherein the one or more dermal fillers are included in the one or more microneedles 30 and the one or more dermal fillers include hyaluronic acid, optionally with various viscosities and cross-linking.

Clause 7. The system 1 of any of the preceding Clauses, wherein the one or more medications 21, drugs, active ingredients are for cosmetic, pain management and/or other therapeutic purposes.

Clause 8. The system 1 of any of the proceeding Clauses, wherein the one or more trays 10 each carry one or more of the substrates thereon.

Clause 9. The system 1 of any of Clauses 1 to 7, wherein the one or more substrates are one or more adhesive patches 20 and free of the one or more trays 10.

Clause 10. The system 1 of any of the preceding Clauses, wherein the one or more substrates each carry the one or more microneedles 30.

Clause 11. The system 1 of any of the preceding Clauses, wherein the one or more microneedles 30 are configured with a regular shape and diverse geometry which is substantially a pencil point, spiral, quincke, atraucan, sprotte, arrowhead, turret, triangular, conical, pyramidal the like, or different, or any combination thereof for any single substrate.

Clause 12. The system 1 of any of the proceeding Clauses, wherein each of the one or more microneedles 30 is prepackaged with a lyophilized powder or quantified Botulinum toxin purified protein units and a corresponding amount of preservative free 0.9% or similar saline at predetermined temperatures which preserve the integrity and function of the Botulinum toxin purified protein.

Clause 13. The system 1 of any of the preceding Clauses, wherein the one or more microneedles 30 are free of any additional carrier separate from a frozen saline, or other reconstitution sterile fluids or stabilizing agents for the neurotoxin (i.e., to maintain an accurate dilution).

Clause 14. The system 1 of any of the preceding Clauses, wherein a shape, depth, and/or size of the plurality of microneedles is elected based on a target intradermal and/or subdermal location for delivering and dissolution of the medication.

Clause 15. The system 1 of any of the proceeding Clauses, wherein the one or more microneedles 30 on a substrate and/or a tray 10 are in a plurality of differing shapes, sizes, consistency, frozen vs. polymeric.

Clause 16. The system 1 of Clause 15, wherein the differing shapes include two to three or even more geometry shapes, e.g. triangular, conical, spiral, pyramidal, and/or sizes.

Clause 17. The system 1 of any of the preceding Clauses, wherein the one or more microneedles 30 are configured in one or more arrays, groups, random patterns, the like, or any combination thereof on the one or more substrates, trays 10, or both.

Clause 18. The system 1 of any of the preceding Clauses, wherein the one or more microneedles 30 are configured such as to entirely, or mostly, penetrate through an epidermis and allow release of the one or more medications 21 and mixing with the one or more carriers at target level as the one or more carrier; e.g., saline, becomes fluid at body temperature.

Clause 19. The system 1 of any of the preceding Clauses, wherein the system 1 is configured for safe patient self-administration under virtual and/or remote medical guidance.

Clause 20. The system 1 of any of the preceding Clauses, wherein the one or more microneedles 30 include a plurality of layers of microneedles.

Clause 21. The system 1 of Clause 20, wherein the plurality of layers of microneedles are nesting microneedles 40, non-nesting microneedles, or both.

Clause 22. The system 1 of any of the preceding Clauses, wherein the plurality of microneedles includes a plurality of nesting microneedles 40 in overlapping layers.

Clause 23. The system 1 of Clause 22, wherein the plurality of nesting microneedles 40 have an increasing width and length from the leading top, layer on, therefore offering a longer piercing depth than a single microneedle.

Clause 24. The system 1 of any of Clauses 20 to 23, wherein the plurality of microneedles include one or more leading microneedles 42, one or more intermediate microneedles 44, one or more trailing microneedles 46, or a combination thereof.

Clause 25. The system 1 of any of Clauses 20 to 24, wherein the plurality of microneedles may each have a length of about 50 microns to about 2,000 microns or more.

Clause 26. The system 1 of any of Clauses 20 to 25, wherein one or more leading microneedles 42 each have a length of about 50 microns to about 700 microns and more robust structure to mechanically overcome tension at stratum corneum and ensure complete skin penetration.

Clause 27. The system 1 of any of Clauses 20 to 26, wherein one or more intermediate microneedles 44, trailing microneedles 46, or both may have length of about 100 microns to about 1500, or to 2000 microns or more.

Clause 28. The system 1 of any of the preceding Clauses, wherein the plurality of microneedles 30 are nesting microneedles 40 which are formed such that a tip 3 of an intermediate microneedle 44 and/or trailing microneedle 46, from a subsequent layer, is embedded in a base 4 of a leading microneedle 42 from the top layer, and/or intermediate microneedle 44. Frozen microneedles 30 are not in continuity with polymeric microneedles 34 but nesting within each other.

Clause 29. The system 1 of any of Clauses 20 to 28, wherein a shape, a thickness, a length, and/or a mechanical resistance of the leading microneedle 42 will follow Young's modulus to ensure penetration through the stratum corneum.

Clause 30. The system 1 of any of Clauses 20 to 29, wherein an intermediate microneedle 44, trailing microneedle 46, or both can have less mechanical resistance than the leading microneedle 42.

Clause 31. The system 1 of any of Clauses 20-30, wherein the tips 3 of frozen or polymeric microneedles 30, 34 have a predetermined rate of dissolution. This rate can be anticipated before further nesting microneedles 40 layers are advanced through the open micropores in the stratum corneum.

Clause 32. The system 1 of any of Clauses 20 to 31, wherein at least one of the one or more microneedles 30 is free of any medication except for one or more carriers.

Clause 33. The system 1 of any of Clauses 20 to 32, wherein one or more leading microneedles 42 are free of any medication except for one or more carriers and/or one or more analgesics/local anesthetics.

Clause 34. The system 1 of any of Clauses 20 to 33, wherein the one or more leading, intermediate, and/or trailing microneedles 42, 44, 46, respectively, include the one or more neurotoxins or recombinant neurotoxin.

Clause 35. The system 1 of Clause 34, wherein the one or more neurotoxins includes BTPP type A.

Clause 36. The system 1 of Clause 35, wherein the BTPP is in the amount of about 0.05 units to about 100 units per microneedle.

Clause 37. The system 1 of Clause 21 to 36, wherein the BTPP is in the amount of about 0.05 units to about 2 units per microneedle.

Clause 38. The system 1 of any of Clauses 20 to 37, wherein the one or more leading, intermediate, and/or trailing microneedles 42, 44, 46, respectively, include one or more dermal fillers.

Clause 39. The system 1 of Clause 38, wherein the one or more dermal fillers include hyaluronic acid.

Clause 40. The system 1 of Clause 39, wherein the HA filler comprises HA fillers of different viscosities and cross linking.

Clause 41. The system 1 of Clause 39 or 40, wherein the hyaluronic acid is about 0.025 ml to about 2 ml per microneedle.

Clause 42. The system 1 of any of Clauses 20 to 41, wherein the one or more leading, intermediate, and/or trailing microneedles 42, 44, 46, respectively, include one or more analgesics or local anesthetics.

Clause 43. The system 1 of Clause 42, wherein the one or more local anesthetics include lidocaine, articaine, tetracaine, mepivacaine, prilocaine the like, or a combination thereof.

Clause 44. The system 1 of Clause 42 or 43, wherein the one or local anesthetics include the lidocaine 1%.

Clause 45. The system 1 of Clause 44, wherein the lidocaine 1% is provided in the system 1 at a total volume of about 0.45 ml or less per microneedle.

Clause 46. The system 1 of any of Clauses 20 to 45, wherein the one or more carriers include a saline.

Clause 47. The system 1 of any of Clauses 20 to 46, wherein the saline is in the amount of about 0.00125 ml to about 0.3 ml per microneedle.

Clause 48. The system 1 of any of Clauses 20 to 47, wherein the one or more carriers is in the amount of 0.05 ml to 3 ml per 1 unit of the one or more neurotoxins.

Clause 49. The system 1 of any of Clauses 20 to 48 wherein the one or more carriers is in the amount of about 0.1 ml per 1 ml of the one or more dermal fillers.

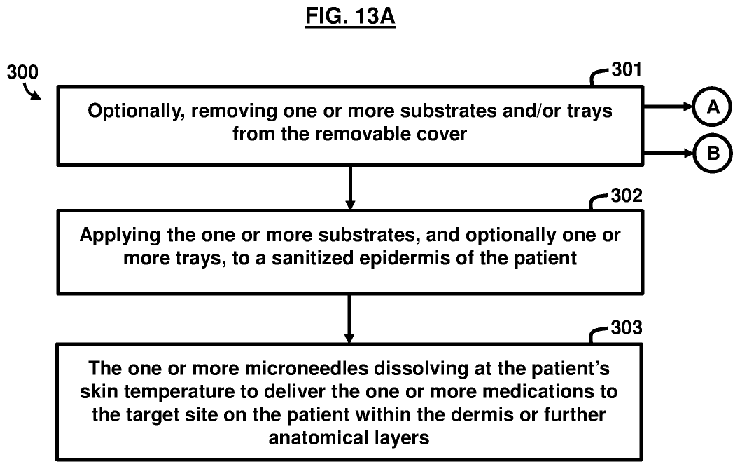

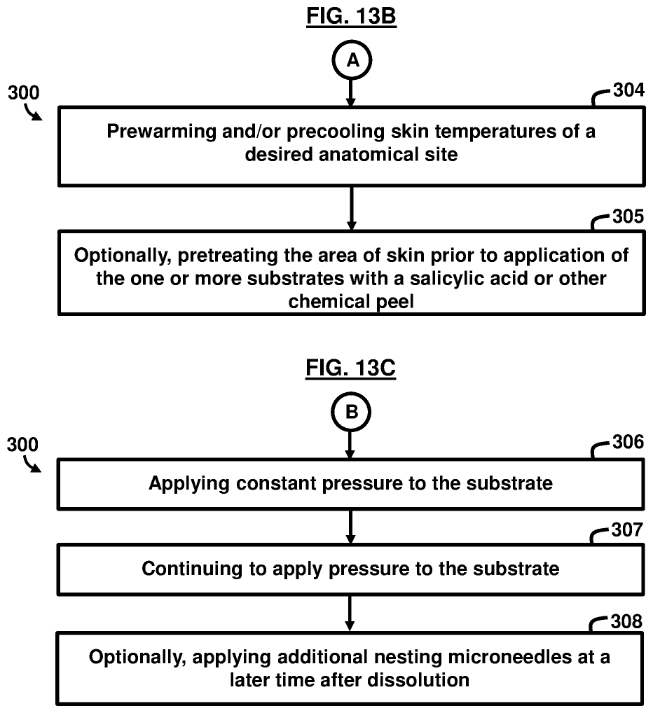

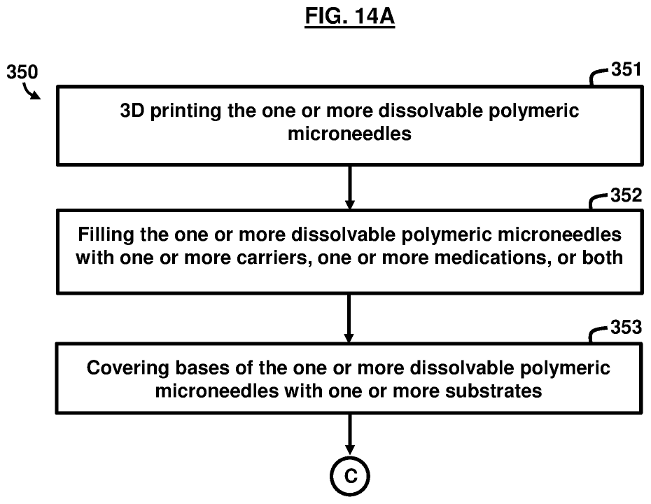

Clause 50. A method 300 (shown in FIGS. 13A through 13C, with reference to FIGS. 1 through 12) of applying one or more medications 21 of the system 1 of any of the preceding Clauses.

Clause 51. The method 300 of Clause 50 comprising: a) optionally, removing (301) one or more substrates and/or trays 10 from the removable cover; b) applying (302) the one or more substrates, and optionally one or more trays 10, to a sanitized epidermis of the patient such that the one or more microneedles 30 penetrate through an epidermis to a dermis level or deeper; and c) the one or more microneedles 30 dissolving (303) at the patient's skin temperature to deliver the one or more medications 21 to the target site on the patient within the dermis or further anatomical layers.

Clause 52. The method 300 of Clause 51 further comprising: a) prewarming and/or precooling (304) skin temperatures of a desired anatomical site to apply the one or more substrates with an infrared light, a warm compress, a cool compress, ice, a gel pack, and/or other suitable methods; and b) optionally, pretreating (305) the area of skin prior to application of the one or more substrates with a salicylic acid or other chemical peel to disrupt cellular junctions in the epidermis and facilitate penetration of the one or more microneedles 30.

Clause 53. The method 300 of Clause 51 or 52 further comprising: a) applying (306) constant pressure to the substrate, wherein the substrate carries a plurality of nesting microneedles 40, such that a first layer of leading microneedles 42 penetrates into the stratum corneum, dissolves, and forms a plurality of microchannels; b) continuing (307) to apply pressure to the substrate such that one or more subsequent layers of nesting intermediate microneedles 44 and/or trailing microneedles 46 advance through the microchannels and into the dermis or even deeper (e.g., intramuscular); and c) optionally, applying (308) additional nesting microneedles 40 (for example, applying additional patches 20 of a subsequent system 1) at a later time (e.g., 3 months) after dissolution.

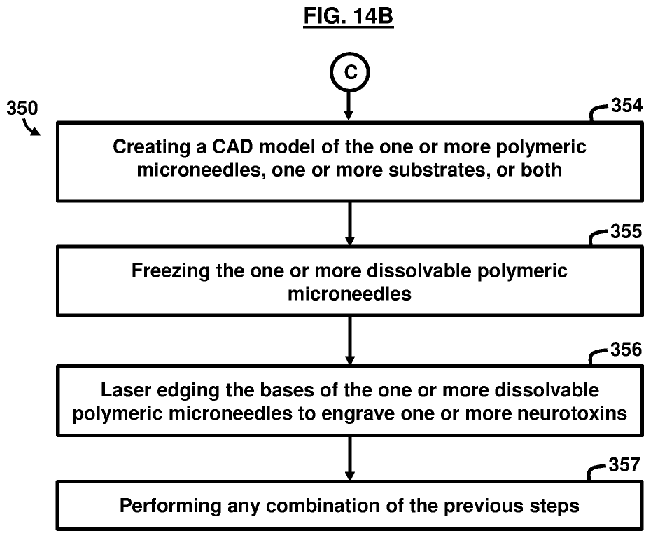

Clause 54. A method 350 of manufacturing (as shown in FIGS. 14A and 14B, with reference to FIGS. 1 through 13C) one or more dissolvable polymeric microneedles 34 comprising the steps of: a) 3D printing (351) the one or more dissolvable polymeric microneedles 34 which may be hollow, made of x-HA (cross-linked) with medium and low molecular weight HA; MeHA(methacrylic anhydride cross linked HA) microneedles in particular have increased stiffness and can easily be used in 3D printing; b) filling (352) the one or more dissolvable polymeric microneedles 34 with one or more carriers, one or more medications 21, or both; and c) covering (353) bases 4 of the one or more dissolvable polymeric microneedles 34 with one or more substrates.

Clause 55. The method 350 of Clause 54 wherein the method 350 includes the steps of: a) creating (354) a CAD model of the one or more polymeric microneedles 34, one or more substrates, or both; b) freezing (355) the one or more dissolvable polymeric microneedles 34; c) laser edging (356) the bases 4 of the one or more dissolvable polymeric microneedles 34 to engrave one or more neurotoxins which may be in powder form (lyophilized) or liquid form; or d) performing (357) any combination of the previous steps.

Clause 56. The method 350 of Clause 54 or 55 wherein the 3D printing is two photon polymerization (TPP) 3D printing.

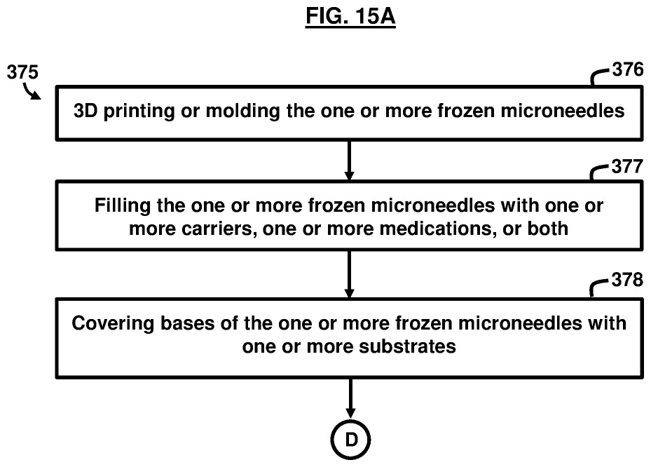

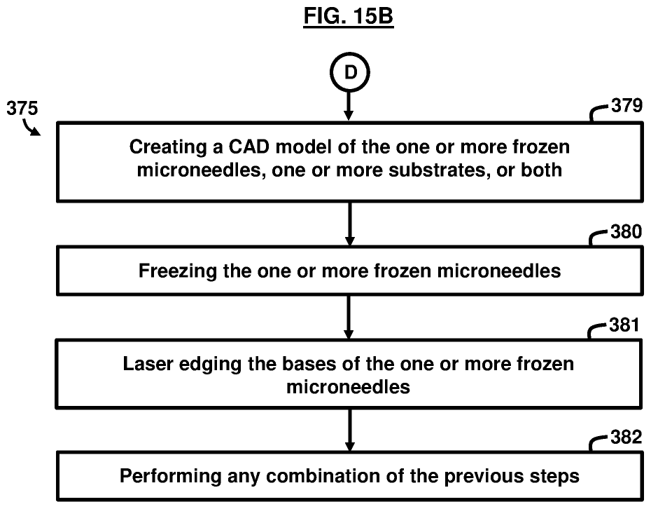

Clause 57. A method 375 of manufacturing (as shown in FIGS. 15A and 15B, with reference to FIGS. 1 through 14B) one or more frozen microneedles 30 comprising the steps of: 3D printing or molding (376) the one or more frozen microneedles 30 which may be hollow; filling (377) the one or more frozen microneedles 30 with one or more carriers, one or more medications 21, or both; and covering (378) bases 4 of the one or more frozen microneedles 30 with one or more substrates (e.g., patches 20).

Clause 58. The method 375 of Clause 57 wherein the method 375 includes the steps of: creating (379) a CAD model of the one or more frozen microneedles 30, one or more substrates, or both; freezing (380) the one or more frozen microneedles 30; laser edging (381) the bases 4 of the one or more frozen microneedles 30 to engrave one or more neurotoxins which may be in powder form or liquid form; or performing (382) any combination of the previous steps.

Clause 59. The method 375 of Clause 57 or 58 wherein the 3D printing is ice 3D printing.

Clause 60. The methods 350, 375 of Clauses 54-59 wherein the method 350 of manufacturing one or more dissolvable polymeric microneedles 34 and the method 375 of manufacturing one or more frozen microneedles 30 are performed sequentially, in parallel, or any combination thereof.

Clause 61. The methods 350, 375 of Clauses 54-59 wherein the one or more dissolvable polymeric microneedles 34, one or more frozen microneedles 30, or both are produced in batches of 10 to 50 microneedles per microneedle array.

Clause 62. The methods 350, 375 of Clauses 54-59 wherein the one or more dissolvable polymeric microneedles 34, one or more frozen microneedles 30, or both contain a total of 50 to 100 units of the one or more neurotoxins per substrate.

Clause 63. A system 1 for delivering one or more ingredients through an epidermis of a subject, wherein the system 1 includes: a tray 10, one or more substrates (e.g., patches 20) arranged on the tray 10, the substrates carrying one or more microneedle assemblies, wherein the microneedle assemblies comprise a plurality of nested microneedles wherein a tip 3 of one of the microneedles of the plurality of microneedles 30 is arranged in a body of another one of the plurality of microneedles assemblies, and wherein the microneedle assemblies are dissolvable and configured for insertion into a dermis via insertion through an epidermis of the patient's skin and wherein one or more of the microneedles carry the one or more ingredients for delivery of into the dermis and/or further layers.

Clause 64. The system 1 of Clause 63, wherein the ingredients are included in the one or more microneedles 30 as coated tips 65, lyophilized powder laser engraved in the base 4 or embedded in microspheres of poly-(L-amide) or cross-linked hyaluronic acid within a hollow portion of a body of the microneedles.

Clause 65. The system 1 of Clause 63 or 64, wherein the ingredients of the microneedles 30 include: one or more neurotoxins or recombinant neurotoxin and/or one or more dermal fillers; and one or more carriers.

Clause 66. The system 1 of Clause 65, wherein the one or more carriers include a sterile saline solution, and optionally wherein the sterile saline solution is preservative free 0.9% saline.

Clause 67. The system 1 of any of Clauses 63-66, wherein one or more of the microneedles 30 comprises a lyophilized powder or quantified Botulinum toxin type A purified protein units and a corresponding amount of preservative free 0.9% or similar saline.

Clause 68. The system 1 of any of Clauses 63-67, wherein the ingredients of the microneedles 30 include one or more analgesics or local anesthetics.

Clause 69. The system 1 of any of Clauses 63-68, wherein the base 4 includes one or more adhesive patches 20.

Clause 70. The system 1 of any of Clauses 63-69, wherein one or more of the microneedles 30 include a hollow interior, wherein the hollow interior contains one or more of the ingredients.

Clause 71. The system 1 of any of Clauses 63-70, wherein one or more of the microneedles 30 include a reservoir containing one or more of the ingredients, and wherein the reservoir is in communication with a hollow shaft of the corresponding microneedle 30 and/or with one or more of the substrates.

Clause 72. The system 1 of any of Clauses 63-71, wherein one or more of the microneedles 30 have a length of about 50 microns to about 2,000 microns.

Clause 73. The system 1 of any of Clauses 63-72, wherein the microneedle assemblies include at least a leading microneedle 42 and a trailing microneedle 46.

Clause 74. The system 1 of Clause 73, wherein the microneedle assemblies include an intermediate microneedle 44, wherein the intermediate microneedle 44 is nested in the leading microneedle 42 and wherein the trailing microneedle 46 is nested in the intermediate microneedle 44.

Clause 75. The system 1 of Clauses 73 or 74, wherein the microneedles 30 of one or more of the microneedle assemblies increase in width and/or length from the leading microneedle 42 to the trailing microneedle 46.

Clause 76. The system 1 of any of Clauses 73-75, wherein one or more of the leading microneedles 42 have a different shape and/or size from the other microneedles of at least one of the microneedle assemblies, and wherein the leading microneedles 42 are configured with a mechanical strength to penetrate a stratum corneum of the skin of the subject.

Clause 77. The system 1 of any of Clauses 73-76, wherein one or more of the intermediate or trailing microneedles 44, 46, respectively, have less mechanical strength than the leading microneedle 42 of the microneedle assembly.

Clause 78. The system 1 of any of Clauses 72-77, wherein one or more of the leading microneedles 42 does not carry the ingredients or only comprises one or more carriers or only comprises one or more analgesics or local anesthetics.

Clause 79. The system 1 of any of Clauses 72-78, wherein one or more of the trailing or intermediate microneedles 46, 44, respectively, have length of about 100 microns to about 1500, or to 2000 microns or more.

Clause 80. The system 1 of any of Clauses 63-79, wherein the microneedles 30 comprise frozen microneedles 30, and/or polymeric microneedles 34.

Clause 81. The system 1 of any of Clauses 63-80, comprising a plurality of substrates carrying microneedles 30.

Clause 82. The system 1 of any of Clauses 63-81, wherein the system 1 is configured for patient self-administration.

Clause 83. A system 1 for delivering one or more medications (e.g., medicament 21) through an epidermis of a patient, wherein the system 1 includes: a) one or more microneedles 30, 34 which are dissolvable and configured for insertion into a dermis via insertion through an epidermis of the patient's skin and delivery of the one or more medications (e.g., medicament 21), drugs, active ingredients into the dermis and/or even further layers of the skin, wherein the one or more microneedles 30, 34 include: one or more neurotoxins or recombinant neurotoxin and/or one or more dermal fillers; one or more carriers; optionally, one or more analgesics or local anesthetics; b) one or more substrates (e.g., patches 20) configured to be temporarily applied onto the epidermis of the patient, wherein the one or more substrates (e.g., patches 20) carry the plurality of microneedles 30, 34 thereon; c) optionally, one or more trays 10 affixed to the one or more substrates (e.g., patches 20) on an opposite side as the one or more microneedles 30, 34, wherein the one or more trays 10 include one or more adhesive patches 20; and d) optionally, one or more removable covers which are removably adhered to the one or more substrates (e.g., patches 20) and/or one or more trays 10.

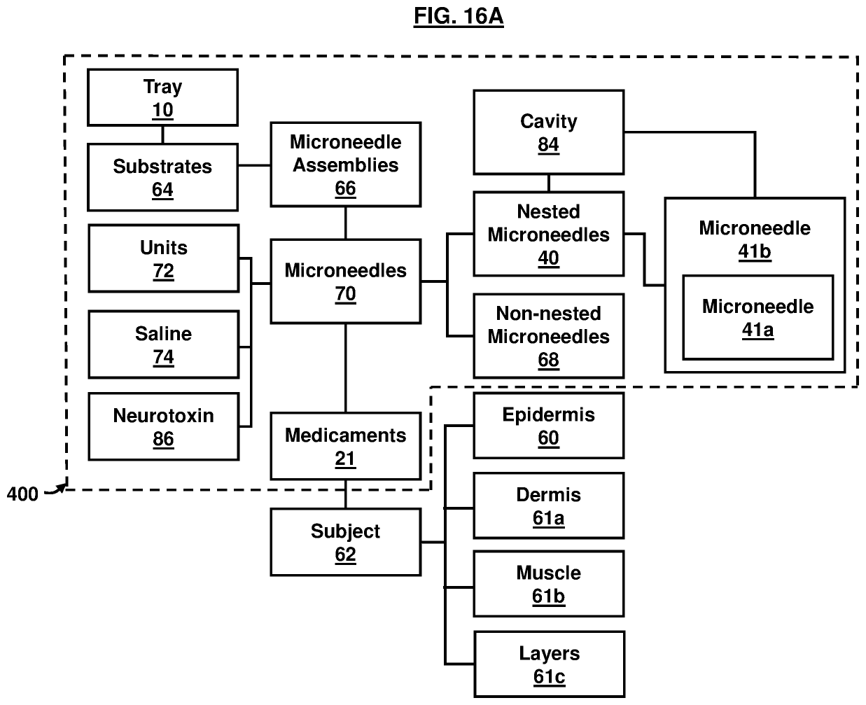

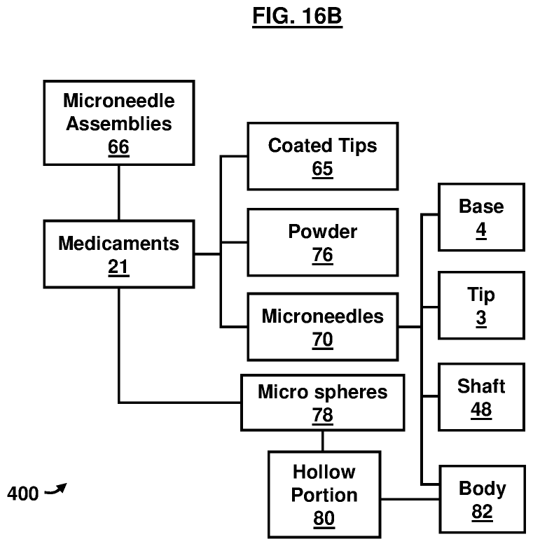

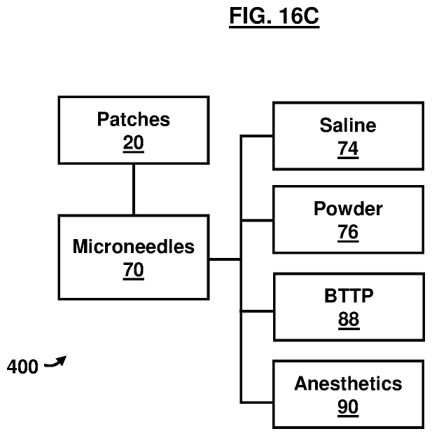

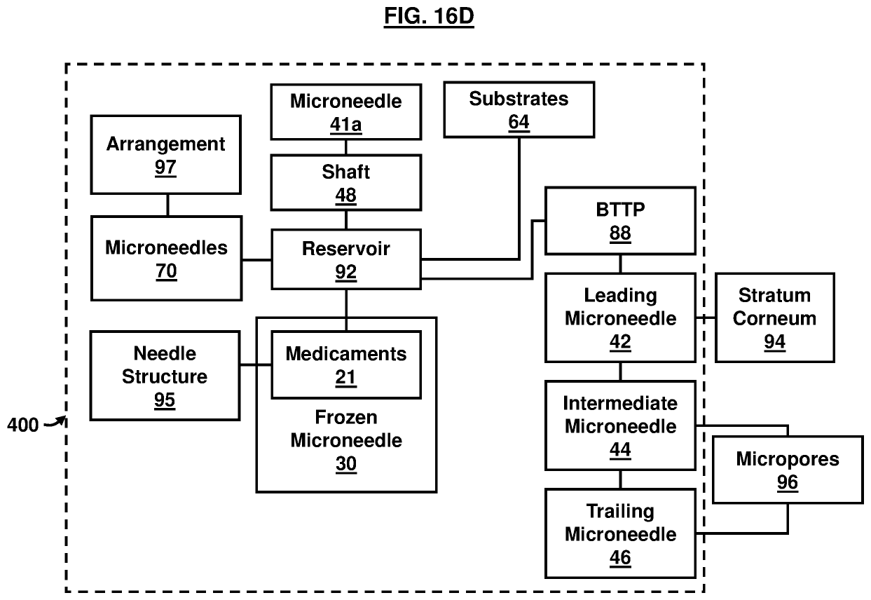

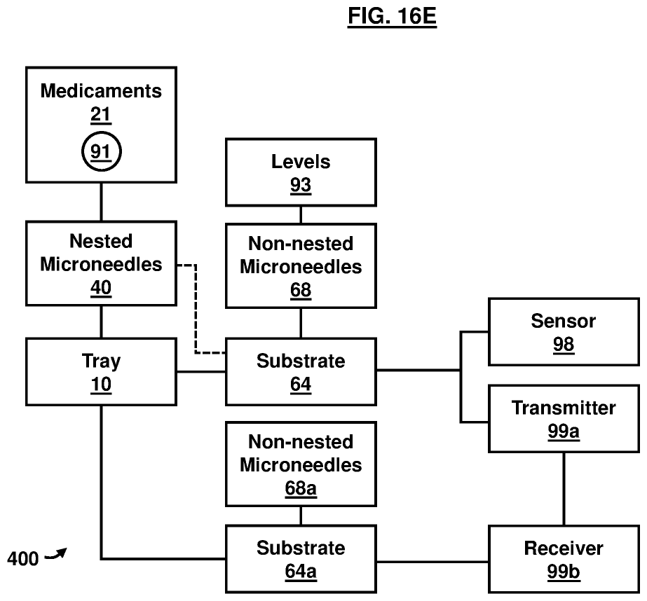

FIGS. 16A through 16E, with reference to FIGS. 1 through 15B, are block diagrams illustrating a system 400 for delivering one or more medications 21 through an epidermis 60 of a subject 62 intradermal or subdermal. The system 400 comprises: a tray 10, and one or more substrates 64 arranged on the tray 10, the substrates 64 carrying one or more microneedle assemblies 66, wherein the one or more microneedle assemblies 66 comprise one or more microneedles 70 configured as a plurality of overlapping 1-6 layers of a combination of non-nested and nested microneedles 68, 40, wherein a nested microneedle 40 comprises one microneedle 41a resting partially within another microneedle 41b, wherein the one or more microneedle assemblies 66 are dissolvable frozen and/or polymeric and are to be inserted into a dermis 61a or muscle 61b of the subject 62 via insertion through an epidermis 60 of the skin of the subject 62, wherein the one or more microneedle assemblies 66 are prepackaged with neurotoxin quantified units 72 with a predetermined reconstitution amount of saline 74 per microneedle 70 to yield a set dilution, and wherein the one or more of the microneedle assemblies 66 carry the one or more medications 21 for delivery of into the dermis 61a and/or further layers 61c of the subject 62.

The one or more medications 21 may be included in the one or more microneedles assemblies 66 as coated tips 65, lyophilized powder 76 laser engraved in a base 4, tip 3, or shaft 48 of the one or more microneedles 70, or embedded in micro spheres 78 of poly-(L-amide) or cross-linked hyaluronic acid within a hollow portion 80 of a body 82 of the one or more microneedles 70. The nested microneedle 40 may comprise a nesting cavity 84 that at least partially receives and/or retains a subsequent microneedle 41b inserted therein. The one or more microneedles 70 may comprise: one or more neurotoxins or recombinant neurotoxin 86; and preservative free saline 74. Each of the one or more microneedles 70 may comprise a lyophilized powder 76 or quantified Botulinum toxin purified protein type A (BTTP) 88 and a preservative free, frozen saline 74. The one or more microneedles 70 may be free of any additional carrier separate from a frozen saline 74. The one or more microneedles 70 may comprise one or more local anesthetics 90 selected from the group consisting of lidocaine, prilocaine, mepivacaine. The one or more microneedles 70 may comprise a base 4 comprising one or more adhesive peel off patches 20. The one or more microneedles 70 may comprise a hollow interior (e.g., hollow portion 80) containing one or more of the medications 21.

The one or more microneedles 70 may comprise a reservoir 92 containing one or more of the medications 21, wherein the reservoir 92 is in communication with a hollow shaft 48 of a corresponding microneedle 41a and/or with the one or more substrates 64, and wherein the reservoir 92 is to propel BTTP embedded micro-spheres 88 through fluidic force and/or a pressure leading microneedle 42 through a stratum corneum 94 and intermediate or trailing microneedles 44, 46 through created micropores 96.

The one or more microneedle assemblies 66 may comprise a leading microneedle 42, one or more intermediate microneedles 44, and one or more trailing microneedles 46, or a combination thereof, wherein the one or more intermediate microneedles 44 is nested in the leading microneedle 42, and wherein the one or more trailing microneedles 46 is nested in the one or more intermediate microneedles 44. One aspect of using nested microneedles 40 over non-nested microneedles 68 relates to the possibility of having dissolvable microneedles nesting with hollow microneedles or hydrogels like microneedles. In this manner the leading microneedle 42 can be dissolvable, the intermediate microneedle 44 can be hydrogel forming, and the trailing microneedle 46 hollow connected to the reservoir 92.

The leading microneedle 42 may be triangular shape, the one or more intermediate microneedles 44 may be pyramidal shape, and the one or more trailing microneedles 46 may be conical shape, in an example. The tip 3 of the one or more intermediate microneedles 44 and/or the one or more trailing microneedles 46 may be embedded in a base 4 of the leading microneedle 42. The one or more medications 21 in frozen microneedles 30 may form a needle structure 95 without any additional carriers. The mechanical resistance of the one or more intermediate microneedles 44 and the one or more trailing microneedles 46 may be less than the mechanical resistance of the leading microneedle 42. The microneedles 70 of the one or more microneedle assemblies 66 may progressively increase in width and/or length from the leading microneedle 42 to the one or more trailing microneedles 46, as a whole along an entire height of a connected assembly 66, and wherein the nested microneedle 40 is shorter than an overall combination of overlapping microneedles 70. The one or more microneedles 70 may be provided as a plurality of microneedles 70 and arranged in one or more arrays, groups, random patterns (e.g., arrangement 97), or a combination thereof on the tray 10, the one or more substrates 64, or a combination thereof.

The non-nested microneedles 68 may be configured to (i) determine glucose or viral RNA levels 93 at an intercellular level, through a continuous sensor 98 and transmitter 99a incorporated within a substrate 64 of a non-nested microneedle 68; (ii) transmit information about the glucose or viral RNA levels 93 to a receiver 99b incorporated in another substrate 64a of another non-nested microneedle 68a, on the tray 10 that supports the nested microneedles 40 with targeted medications (e.g., medicaments 21) comprising short-acting insulin, long-acting insulin, or Covid-19 antibodies (collectively 91). In another example, the non-nested microneedles 98 may be configured on the same substrate 64 as the nested microneedles 40.

In an example, the trailing microneedles 46 advance through the intermediate microneedles 44 and the intermediate microneedles 44 advance through leading microneedles 42. Calculating the rate of dissolution for each layer of microneedles 30 in the nesting assembly can determine with accuracy the rate of delivery for each medication. In some examples, this could have applications in delivering insulin like medications for diabetes, or pain killers. Non-nesting microneedles 68 can function to determine the blood level of certain parameters as glucose or covid related viruses while nesting microneedles 40 on the same substrate 64 or different substrate 64a, but the same tray 10, can timely deliver medications and targeted therapies based on the levels detected by the non-nesting microneedles 68. For example, a set of geometrically identical or different non-nesting microneedles 68, 68a on one substrate 64 equipped with a continuous glucose sensor 98 or continuous RNA viral level detection incorporating wireless transmission technology such as Bluetooth® technology, can estimate glucose levels 93 in the fluid between epidermal and dermal cells. The substrate 64 may be equipped with a transmitter 99a which sends the information to a receiver 99b incorporated in same substrate 64 or another substrate 64a, on the tray 10 that supports nesting microneedles 10 with targeted medications 21 like short acting, long-acting insulin, or respectively Covid-19 antibodies (collectively 91).

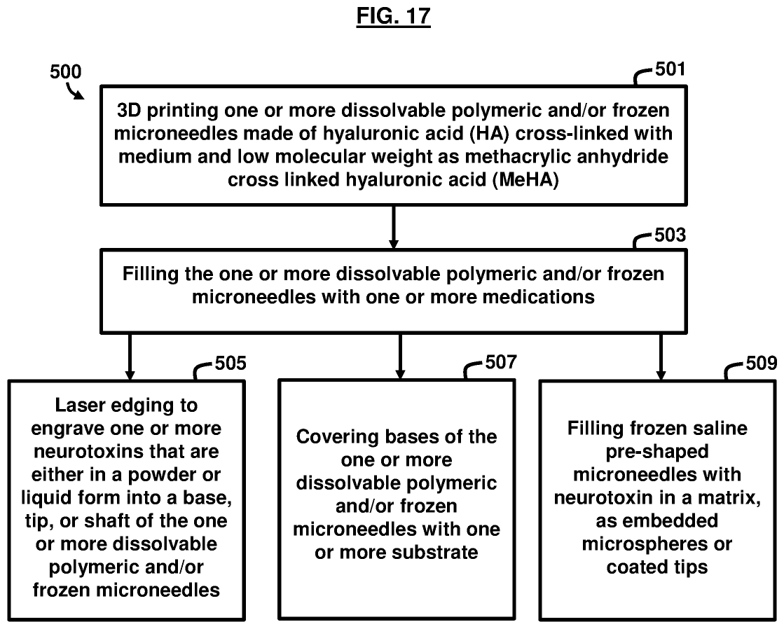

FIG. 17, with reference to FIGS. 1 through 16E, is a flow diagram illustrating a method 500 of manufacturing one or more dissolvable polymeric and/or frozen microneedles 34, 30, the method 500 comprising 3D printing (501) one or more dissolvable polymeric and/or frozen microneedles 34, 30 made of hyaluronic acid (HA) cross-linked with medium and low molecular weight as methacrylic anhydride cross linked hyaluronic acid (MeHA); filling (503) the one or more dissolvable polymeric and/or frozen microneedles 34, 30 with one or more medications 21; and performing: laser edging (505) to engrave one or more neurotoxins 86 that are either in a powder or liquid form into a base 4, tip 3, or shaft 48 of the one or more dissolvable polymeric and/or frozen microneedles 34, 30; covering (507) bases 4 of the one or more dissolvable polymeric and/or frozen microneedles 34, 30 with one or more substrate 64; or filling (509) frozen saline pre-shaped microneedles 30 with neurotoxin 86 in a matrix, as embedded microspheres or coated tips 65.

The foregoing description of the specific embodiments will so fully reveal the general nature of the embodiments herein that others may, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein may be practiced with modification within the spirit and scope of the appended claims.

What it is claimed:

1. A system for delivering one or more medications through an epidermis of a subject intradermal or subdermal, wherein the system comprises:
a tray, and
one or more substrates arranged on the tray, the substrates carrying one or more microneedle assemblies,
wherein the one or more microneedle assemblies comprise one or more microneedles configured as a plurality of overlapping 1-6 layers of a combination of non-nested and nested microneedles,
wherein a nested microneedle comprises one microneedle resting partially within another microneedle,
wherein the one or more microneedle assemblies are dissolvable frozen and/or polymeric and are to be inserted into a dermis or muscle of the subject via insertion through an epidermis of the subject,
wherein the one or more microneedle assemblies are prepackaged with neurotoxin quantified units with a predetermined reconstitution amount of saline per microneedle to yield a set dilution, and
wherein the one or more of the microneedle assemblies carry the one or more medications for delivery of into the dermis and/or further layers of the subject.

2. The system of claim 1, wherein the one or more medications are included in a microneedle as coated tips, lyophilized powder laser engraved in a base, tip, or shaft of the microneedle, or embedded in micro spheres of poly-(L-amide) or cross-linked hyaluronic acid within a hollow portion of a body of the one or more microneedles.

3. The system of claim 1, wherein the nested microneedle comprises a nesting cavity that at least partially receives and/or retains a subsequent microneedle inserted therein.

4. The system of claim 3, wherein each of the one or more microneedles comprises a lyophilized powder or quantified Botulinum toxin purified protein type A (BTTP) and a preservative free, frozen saline.

5. The system of claim 1, wherein the one or more microneedles are free of any additional carrier separate from a frozen saline.

6. The system of claim 1, wherein the one or more microneedles comprise one or more local anesthetics selected from the group consisting of lidocaine, prilocaine, mepivacaine.

7. The system of claim 1, wherein the one or more microneedles comprises a base comprising one or more adhesive peel off patches.

8. The system of claim 1, wherein the one or more microneedles comprise a hollow interior containing one or more of the medications.

9. The system of claim 1, wherein the one or more microneedles comprises a reservoir containing one or more of the medications, wherein the reservoir is in communication with a hollow shaft of a corresponding microneedle and/or with the one or more substrates, and wherein the reservoir is to propel BTTP embedded micro-spheres through fluidic force and/or a pressure leading microneedle through a stratum corneum and intermediate or trailing microneedles through created micropores.

10. The system of claim 1, wherein the one or more microneedle assemblies comprise a leading microneedle, one or more intermediate microneedles, and one or more trailing microneedles, or a combination thereof, wherein the one or more intermediate microneedles is nested in the leading microneedle, and wherein the one or more trailing microneedles is nested in the one or more intermediate microneedles.

11. The system of claim 10, wherein the leading microneedle is triangular shape, the one or more intermediate microneedles is pyramidal shape, and the one or more trailing microneedles is conical shape.

12. The system of claim 10, wherein a tip of the one or more intermediate microneedles and/or the one or more trailing microneedles is embedded in a base of the leading microneedle.

13. The system of claim 10, wherein the one or more medications in frozen microneedles form a needle structure without any additional carriers.

14. The system of claim 10, wherein the microneedles of the one or more microneedle assemblies progressively increase in width and/or length from the leading microneedle to the one or more trailing microneedles, as a whole along an entire height of a connected assembly, and wherein each of the nested microneedles is shorter than an overall combination of overlapping microneedles.

15. The system of claim 1, wherein the one or more microneedles are provided as a plurality of microneedles and arranged in one or more arrays, groups, random patterns, or a combination thereof on the tray, the one or more substrates, or a combination thereof.

16. A method of delivering a medicament in liquid suspension, wherein the method comprises:
removing one or more adhesive patches from a tray, wherein the one or more adhesive patches carry one or more microneedles;
applying the one or more adhesive patches containing the one or more microneedles to an epidermis of a subject, wherein the one or more microneedles comprise a plurality of overlapping 1-6 layers of a combination of non-nested and nested microneedles, and wherein a nested microneedle comprises one microneedle resting partially within another microneedle;
inserting, into a dermis of the subject, the one or more microneedles formed of the liquid suspension containing the medicament, wherein the one or more microneedles are in a frozen form and/or polymeric upon insertion; and
the one or more microneedles melting after insertion into the dermis.

17. The method of claim 16, comprising applying the one or more patches in an overlapping pattern on the epidermis of the subject.

18. The method of claim 16, comprising:
applying pressure to the one or more microneedles upon insertion in the dermis of the subject, wherein a first layer of a leading microneedle dissolves after insertion and forms a plurality of microchannels in the dermis; and
continuing to apply pressure to the one or more microneedles, wherein subsequent layers of intermediate microneedles or trailing microneedles advance through the microchannels.

19. The method of claim 16, wherein the non-nested microneedles are to (i) determine glucose or viral RNA levels at an intercellular level, through a continuous sensor and transmitter incorporated within a substrate of a non-nested microneedle; (ii) transmit information about the glucose or viral RNA levels to a receiver incorporated in another substrate of another non-nested microneedle, on the tray that supports the nested microneedles with targeted medications comprising short-acting insulin, long-acting insulin, or Covid-19 antibodies.

20. A method of manufacturing one or more dissolvable polymeric and/or frozen microneedles, the method comprising:
3D printing one or more dissolvable polymeric and/or frozen microneedles made of hyaluronic acid (HA) cross-linked with medium and low molecular weight as methacrylic anhydride cross linked hyaluronic acid (MeHA);
filling the one or more dissolvable polymeric and/or frozen microneedles with one or more medications; and
performing:
laser edging to engrave one or more neurotoxins that are either in a powder or liquid form into a base, tip, or shaft of the one or more dissolvable polymeric and/or frozen microneedles;
covering bases of the one or more dissolvable polymeric and/or frozen microneedles with one or more substrate; or
filling frozen saline pre-shaped microneedles with neurotoxin in a matrix, as embedded microspheres or coated tips.
